(19) United States
(12) Patent Application Publication
(10) Pub. No.: US 2019/0203230 A1
(43) Pub. Date: Jul. 4, 2019

(54) POROUS MEMBRANE-BASED MACROMOLECULE DELIVERY SYSTEM

(71) Applicant: NOVARTIS AG, Basel (CH)

(72) Inventors: Fabio FACHIN, Cambridge, MA (US); Michael FIORINO, Cambridge, MA (US); Cameron LEE, Cambridge, MA (US); Yi YANG, Cambridge, MA (US); Jonathan YEN, Cambridge, MA (US)

(73) Assignee: Novartis AG, Basel (CH)

(21) Appl. No.: 16/331,967

(22) PCT Filed: Sep. 28, 2017
(86) PCT No.: PCT/US2017/054110

§ 371 Date: Mar. 9, 2019

Related U.S. Application Data
(60) Provisional application No. 62/401,053, filed on Sep. 28, 2016.

(51) Int. Cl. C12N 15/90; C12N 15/87

(52) U.S. Cl. CPC C12N 15/907; C12N 2800/80; C12N 2310/20; C12N 15/87

(57) ABSTRACT

In one aspect, a method of processing a cell is disclosed, which includes passing a cell through a pore of a membrane comprising a plurality of pores while exposing the cell to an agent so as to cause a change in the cell, thereby allowing said agent to enter the cell, where each of said pores extends from an input opening to an output opening and has at least one cross-sectional dimension, and in many embodiments a maximum cross-sectional dimension, less than a diameter of said cell. For example, at least one cross-sectional dimension of the pore, and in many embodiment the maximum cross-sectional dimension of the pore, can be less than about 40 microns, or less than about 30 microns, or less than about 20 microns, or less than about 15 microns, or less than about 10 microns.

### CROSS-REFERENCE TO RELATED APPLICATION

This application claims priority to U.S. Provisional Application No. 62/401,053, filed on Sep. 28, 2016, the content of which is incorporated herein by reference in its entirety.

### FIELD

The present invention relates generally to systems and methods for processing cells, and more particularly to such systems and methods that allow manipulating cells so as to enable the cells to uptake agents, such as biological agents, from an external environment.

### BACKGROUND

A variety of therapeutic and diagnostic applications require the uptake of a variety of agents, e.g., biological agents, by cells. In many cases, the cells are not permeable to such agents and hence their delivery into the cells poses significant challenges. By way of example, gene editing technologies require the delivery of gene editing components into a cell. One such gene editing technology, commonly known as CRISPR (clustered regularly interspaced short palindromic repeats), requires the delivery of ribonucleoprotein (RNP) complexes to cells. However, the delivery of such RNP complexes across a cell's membrane is difficult.

A number of techniques have been developed to facilitate the delivery of foreign agents into cells. Some examples of such techniques include electroporation, optoporation, direct puncture via nanoneedles, etc. These techniques can, however, suffer from a number of shortcomings. For example, they can exhibit low efficiency and/or low cell viability. In some cases, such techniques require the use of special buffers. Moreover, many such techniques are not suitable for parallel processing of a large number of cells.

Accordingly, there is a need for improved systems and methods for delivery of agents into cells.

### SUMMARY

In one aspect, a method of cell processing is disclosed, which comprises passing a plurality of cells through one or more pores of a membrane comprising a plurality of pores while exposing the cells to an agent so as to cause a change in the cells, thereby allowing said agent to enter at least one of the cells, wherein each of said pores extends from an input opening to an output opening and has a maximum cross-sectional dimension in a range of about 7 micrometers (microns) to about 9 microns, and preferably in a range of about 8 microns to about 9 microns. In some embodiments, the maximum cross-sectional dimension of each pore is about 7 microns, or about 8 microns, or about 9 microns. In some embodiments, at least about 80%, or at least about 90%, or at least about 95%, or at least about 99% or more of the pores have a cross-sectional dimension in a desired range or at a specific value, e.g., in a range of about 7 microns to about 9 microns, or in a range of about 8 microns to about 9 microns, or 7 microns, or 8 microns or 9 microns.

In some embodiments, the cells can be circulatory cells. Some examples of suitable circulatory cells include, without limitation, stem cells, progenitor cells, immune effector cells, hematopoietic stem cells, hematopoietic progenitor cells, hematopoietic stem and progenitor cells (HSPCs). In some embodiments, the cells can be CD34+ cells. In other embodiments, the cells can be T cells and/or NK cells. In some embodiments, the cells can be engineered, or can be capable of being engineered, to express a chimeric antigen receptor (CAR). In some embodiments, the cells can be mammalian. In some embodiments, the cells can be human cells.

In some embodiments of the above method for processing circulatory cells, the pores can have a length in a range of about 7 microns to about 10 microns, e.g., in a range of about 8 to 9 microns. In some embodiments, the pores can have a length in a range of about 18 to about 21 microns. In some embodiments, the membrane can have an active surface area in a range of about 7 mm² to about 80 mm², e.g., in a range of about 10 to 20 mm², or in a range of about 30 to about 40 mm², or in a range of about 50 to 60 mm², or in a range of about 70 mm² to about 80 mm². Further, in some such embodiments, the membrane can have a surface pore density in a range of about 1×10⁵ to about 2×10⁶ pores/cm². In some embodiments, the thickness of the membrane can be substantially similar to the lengths of the pores, though in other embodiments thicker membranes can be employed.

In some embodiments of the above method for processing circulatory cells, the cells and the agent can be disposed within a liquid carrier and said liquid carrier can be pushed through said pores via application of a pressure thereto. In some such embodiments, a concentration of the cells within the liquid carrier can be in a range of about 10,000 to about 200,000 cells per microliter. In some embodiments, the concentration of the cells within the liquid carrier can be in a range of about 50,000 to about 200,000 cells per microliter, or in a range of about 100,000 to about 200,000 cells per microliter, or in a range of about 150,000 to about 200,000 cells per microliter, or in a range of about 160,000 to about 200,000 cells per microliter, or in a range of about 170,000 to about 200,000 cells per microliter, or in a range of about 180,000 to about 200,000 cells per microliter, or in a range of about 190,000 to about 200,000 cells per microliter. In some such embodiments, the pressure applied to the liquid carrier can be such that the cells pass through the pores at a rate of at least about 10 to about 20 mL (milliliters) per minute. By way of example, the pressure applied to the liquid carrier can be in a range of about 5 psi to about 20 psi.

In some embodiments, the liquid carrier can include, without limitation, any of water, saline, a basal cell culture medium (e.g., SFEM-II), a serum free medium, and/or HSC brew. In some embodiments, the liquid carrier can include any of polyethylene glycol (PEG), and/or a detergent. In some embodiments, the liquid carrier can further comprise one or more cytokines, one or more growth factors, one or more viability enhancers, and combinations thereof. By way of example, the one or more cytokines can be any of thrombopoietin (TPO), Flt3 ligand (Flt-3L), stem cell factor (SCF), interleukin-6 (IL-6), and combinations thereof.

In some embodiments of the above method for processing circulatory cells, the change caused in the cells via their passage through the pores can be a transient change, e.g., a transient change in the permeability of the cells' membranes.

In some embodiments of the above method for processing circulatory cells, the pores of the membrane can be at least partially coated with polyvinylpyrrolidone.

In some embodiments of the above method for processing circulatory cells, the agent can include a compound to which the cells' membranes are normally impermeable. By way of example, in some embodiments, the agent can be any of a deoxyribonucleic acid (DNA), an ribonucleic acid (RNA), a plasmid, a ribonucleoprotein complex (RNP), a protein, a peptide, a lipid, a polysaccharide, an oligosaccharide, an antisense oligonucleotide, an aptamer, a nanoparticle, a dye, and combinations thereof. By way of the example, the agent can be a gene editing system. Some examples of such gene editing systems can include, without limitation, a CRISPR gene editing system, ZFN gene editing system, TALEN gene editing system, meganuclease gene editing system or Cre recombinase gene editing system. For example, the gene editing system can be a CRISPR gene editing system, where the CRISPR gene editing system includes one or more RNPs, such as Cas9-gRNA complex. By way of example, in some embodiments, the gene editing system can be, e.g., a CRISPR gene editing system, as described in PCT publication WO2017/115268, the contents of which are incorporated herein in their entirety. In other embodiments, the gene editing system can be a gene editing system, e.g., a CRISPR gene editing system, as described in PCT publication WO2017/093969, the contents of which are incorporated herein in their entirety.

In some embodiments, the agent delivered to the cells can comprise a nucleic acid encoding a chimeric antigen receptor (CAR). In some embodiments, the cells can comprise a CAR or nucleic acid encoding a CAR. Chimeric antigen receptors (CARs) and nucleic acids expressing the CARs are described in the following published international applications, the content of each of which is herein incorporated by reference in its entirety: WO2012/07900, WO2014/153270, WO2014/130635, WO2014/130657, WO2015/142675, WO2015/090230, WO2016/014565, WO2016/164731, WO2016/028896, WO2016/014576 and WO2016/014535,

In some embodiments of the above method for processing circulatory cells, the agent can be electrically charged. In other embodiments, the agent can be electrically neutral. In some embodiments, the agent can have a molecular weight greater than about 2 kDa, or greater about 3 k Da, or greater about 10 kDa, or greater about 20 kDa, or greater about 30 kDa, or greater about 40 kDa, or greater than about 50 kDa, or greater than about 60 kDa, or greater than about 70 kDa, or greater than about 80 kDa, or greater than about 90 kDa, or greater than about 100 kDa.

In the above method for processing circulatory cells, the membrane can be formed of a variety of different materials. By way of example, the membrane can include a polymeric material. Some examples of suitable polymeric materials include, without limitation, polycarbonate, polytetrafluoroethylene (PTFE), polystyrene, polyvinylidene fluoride (PVDF), polyethylene terephthalate (PET), poly methyl methacrylate (PMMA), polypropylene (PP), polyimide (PI), cyclic olefin copolymer (COC), cyclo olefin polymer (COP), polyester, and polydimethylsiloxane (PDMS). In some such embodiments, the pores can be formed in the polymeric material via any of ion track etching, laser drilling, plasma etching, or photolithography. In other embodiments, the membrane can be formed of any of a semiconductor, a ceramic, or a metal.

In some of the embodiments of the above method for processing circulatory cells, the pores can have a substantially uniform cross-sectional area along a length of each of the pores. The pores can have a regular or an irregular cross-sectional shape. The pores can have a variety of different shapes. For example, the pores can have a polygonal cross-sectional shape, such as a square, a rectangle, a hexagonal, or an octagonal shape. For example, in some embodiments, the pores can be substantially cylindrical.

In some embodiments of the above method for processing circulatory cells, the pores can have hydrophobic or hydrophilic inner surfaces.

In some embodiments of the above method for processing circulatory cells, the cells can be selected from a collection of heterogeneous cells prior to the step of passing the cells through the membrane. By way of example, the selected cells can have any of a CD3, CD4, CD8, CD27, CD28, CD34, CD90, and CD49f marker.

In some embodiments of the above method for processing circulatory cells, at least about 40%, or at least about 50%, or at least about 60%, or at least about 70%, or at least about 80%, or at least about 90% of the cells uptake said agent via passage through said one or more pores. In some such embodiments, the cells can uptake the agent with a cell viability of greater than about 50%, or greater than about 60%, or greater than about 70%, or greater than about 80%, or greater than about 90%.

In some embodiments of the above method for processing circulatory cells, the membrane can have a maximum thickness that is substantially equal to a maximum length of the pores. By way of example, the pores can have a substantially uniform length and the membrane can have a thickness that is substantially equal to the pore length.

In some embodiments of the above method for processing circulatory cells, the agent can be any of a deoxyribonucleic acid (DNA) (for example, single-stranded or double-stranded DNA), a ribonucleic acid (RNA), a plasmid, a ribonucleoprotein complex (RNP), a protein, a peptide, a lipid, a polysaccharide, an oligosaccharide, an antisense oligonucleotide, an aptamer, a nanoparticle, a dye, a non-membrane permeable compound and combinations thereof.

In some embodiments of the above method for processing circulatory cells, the concentration of the agent in a liquid carrier, which carries the cells and the agent to the porous membrane, can be up to 50 grams/liter.

In some embodiments of the above method for processing circulatory cells, the liquid carrier containing the cells and the agent can be introduced into said pores via an input chamber having a volume less than about 20% of a volume of the liquid carrier introduced into the pores via said input chamber. By way of example, in some embodiments, the liquid carrier containing the cells and the agent can be introduced into the pores via an input chamber having a volume equal to or less than about 10 microliters, e.g., a volume in a range of about 0.5 to about 10 microliters.

In a related aspect, a method of transfecting cells, an in particular circulatory cells, is disclosed, which comprises applying a pressure to a liquid carrier containing a plurality of cells at a cell concentration in a range of about 10,000 to about 200,000 cells per microliter so as to cause the liquid carrier and the cells contained therein to pass through a plurality of pores of one or more porous membranes while exposing the cells to an agent so as to transfect at least some of said cells with said agent at a rate greater than about 2 billion cells, or greater than about 4 billion cells, per minute with a cell viability of at least about 60%, wherein each of said pores has a maximum cross-sectional dimension in a range of about 7 microns to about 9 microns, e.g., in a range of about 8 microns to about 9 microns, or about 7 microns, or about 8 microns, or about 9 microns. In some embodiments of such a method, the pores can have a length in a range of about 7 microns to about 10 microns. In other embodiments, the pores can have a length in a range of about 18 microns to about 21 microns. Further, in some embodiments of such a method, the cell viability after transfection can be at least about 60% or at least about 70%, or at least about 80%, or at least about 90%.

In a related aspect, a system for parallel cell processing is disclosed, which comprises an inlet support element having a plurality of openings for receiving a plurality of samples containing a fluid carrier, a plurality of cells and at least one agent to be internalized by the cells, an outlet support element having a plurality of openings, and a plurality of porous membranes disposed between said inlet support element and said outlet support element and each having a plurality of pores with a maximum cross-sectional dimension less than about 9 microns, e.g., in a range of about 7 microns to about 9 microns, wherein each of the porous membranes is positioned relative to one of the openings in the inlet support element to receive one of the samples and is positioned relative to one of the openings in the outlet support element to allow at least a portion of the sample passing therethrough to reach said outlet opening.

In some embodiments, the system can further include a plurality of meshes each disposed adjacent to one of said porous membranes to provide mechanical support thereto. By way of example, the meshes can be formed of stainless steel.

In some embodiments, the pores can be formed in the polymeric material via any of ion tracking etching, laser drilling, plasma etching, or photolithography.

In some embodiments, the system further includes at least one pressure applicator coupled to at least one of the openings of the input support element for applying a pressure to said samples.

In some embodiments, the system further comprises at least one inlet chamber disposed upstream of said plurality of porous membranes and having an inlet port for receiving said fluid carrier and an outlet port through which said fluid carrier is introduced onto said porous membranes. In some such embodiments, the input chamber has a volume less than about 20% of a volume of the fluid carrier introduced onto said porous membranes. For example, the input chamber can have a volume equal to or less than about 10 microliters, e.g., in a range of about 0.5 to about 10 microliters.

In yet another aspect, a method of processing a cell is disclosed, which includes passing a cell through a pore of a membrane comprising a plurality of pores while exposing the cell to an agent so as to cause a change in the cell, thereby allowing said agent to enter the cell, where each of said pores extends from an input opening to an output opening and has at least one cross-sectional dimension, and in many embodiments a maximum cross-sectional dimension, less than a diameter of said cell. For example, at least one cross-sectional dimension of the pore, and in many embodiments the maximum cross-sectional dimension of the pore, can be less than about 40 microns, or less than about 30 microns, or less than about 20 microns, or less than about 15 microns, or less than about 10 microns. For example, at least one cross-sectional dimension of the pore, and in many embodiments the maximum cross-sectional dimension of the pore, can be in a range of about 2 microns to about 40 microns, or in a range of about 2 microns to about 30 microns, or in a range of about 2 microns to about 20 microns, or in a range of about 2 microns to about 12 microns, or in a range of about 2 microns to about 10 microns, or in a range of about 2 microns to about 10 microns, e.g., in a range of about 5 microns to about 8 microns

In some embodiments, the membrane can have a maximum thickness substantially equal to a maximum length of the pores. In some such embodiments, the pores can have a substantially uniform length and the membrane has a thickness that is substantially equal to that length.

In some cases, the change caused by the passage of a cell through a pore of the membrane can be a transient change. For example, the change (or perturbation) can correspond to a transient change in the permeability of the cell's membrane.

In some embodiments, the porous membrane can have a surface pore density in a range of about 1×10⁵ to about 2×10⁶ pores/cm². In some embodiments, the porous membrane can be at least partially coated with a polymeric material, such as polyvinylpyrrolidone.

In some embodiments, the cell and the agent can be disposed within a liquid carrier and the liquid carrier is pushed through the pores of the membrane via application of a pressure thereto. For example, a pressure of at least about 5 psi, e.g., in a range of about 5 psi to about 100 psi, can be applied to the liquid carrier to generate a flow of the liquid carrier, and consequently the cells and the agent(s) contained therein, through the porous membrane. In some embodiments, the pressure applied to the liquid carrier can cause a flow of the liquid carrier through the pore(s) of the membrane at a flow rate greater than about 10 ml/min (milliliters per minute), e.g., in a range of about 10 ml/min to about 20 ml/min.

A variety of different agents can be introduced into a variety of different cells using the methods according to the present teachings. In many embodiments, the methods according to the present teachings can be employed to deliver into a cell an agent, e.g., a biological compound, to which the cell is normally impermeable. By way of example, the agent can be any of a deoxyribonucleic acid (DNA), a ribonucleic acid (RNA), a plasmid, a ribonucleoprotein complex (RNP), a protein, a peptide, a lipid, a polysaccharide, an oligosaccharide, an antisense oligonucleotide, an aptamer, a nanoparticle, a dye, a non-membrane permeable compound and combinations of the foregoing.

By way of example, in some embodiments, the methods according to the present teachings can be employed to deliver a Cas9-gRNA RNP complex into a cell. In some embodiments, the agent can be electrically charged, while in other embodiments the agent is electrically neutral. The agent can have a variety of different molecular weights. In an aspect, one or more components of a gene editing system, e.g., a CRISPR gene editing system, a zinc finger nuclease gene editing system, a TALEN gene editing system, a meganuclease gene editing system, a Cre recombinase gene editing system, and the like, can be introduced into the cells. In some embodiments, the one or more components of a gene editing system can be one or more components of a CRISPR gene editing system, e.g., comprises or is an RNP comprising a gRNA molecule and a RNA-guided nuclease (such as a Cas9 protein).

In some embodiments, the porous membrane can include a polymeric material, such as polycarbonate, polytetrafluoroethylene (PTFE), polystyrene, polyvinylidene fluoride (PVDF), polyethylene terephthalate (PET), poly methyl methacrylate (PMMA), polypropylene (PP), polyimide (PI), cyclic olefin copolymer (COC), cyclo olefin polymer (COP), polyester, and polydimethylsiloxane (PDMS). The pores can be formed in such a polymeric material, for example, via any of ion track etching, laser drilling, plasma etching, or photolithography.

In some other embodiments, the porous membrane can include any of a semiconductor or a metal.

The methods according to the present teachings can be applied to a variety of different cell types. Some examples of such cells can include progenitor cells, immune effector cells, human embryonic stem cells (hES cells), induced pluripotent stem cells (iPSCs), mesenchymal stem cells, keratinocytes, and human bronchial epithelial cells. For example, the cell can be a T cell.

The pores of the membrane can have a variety of different shapes. For example, the pores can have a regular, or an irregular, cross-sectional shape. Some examples of the regular cross-sectional shape can include, without limitation, a circular, an oval and a polygonal shape, such as a square, a rectangle, a hexagonal, or an octagonal shape. In some embodiments, the pores of a membrane can have a substantially uniform cross-sectional dimension along their lengths. In other embodiments, at least one cross-sectional dimension of the pores can be non-uniform along their lengths.

In some embodiments, the porous membrane can have hydrophilic surfaces. In other embodiments, the porous membrane can have hydrophobic surfaces. For example, the inner surfaces of the pores can be hydrophilic or hydrophobic.

In some embodiments, the above method further includes selecting one or more cells from a collection of heterogeneous cells prior to the step of passing those cells through the pores of the porous membrane. A variety of selection criteria can be used. For example, in some embodiments, the cells are selected based on expression, e.g., surface expression, of any of CD3, CD4, CD8, CD27, CD28, CD34, CD90, CD49f, and combinations thereof.

In a related aspect, a method of transfecting cells is disclosed, which includes passing a fluid carrier containing a plurality of cells and an agent through a porous membrane having a plurality of pores, each of which has a maximum cross-sectional dimension less than a maximum diameter of said cells, such that passage of the cells through the pores causes a physical deformation of the cells sufficient for transfection thereof by said agent. In some embodiments, each of the pores can have an inlet proximate to a top surface of the membrane and an outlet proximate to a bottom surface of the membrane. In some embodiments, the membrane can have a maximum thickness that is substantially equal to a maximum length of the pores. In some embodiments, the pores can have a substantially uniform length and the membrane has a thickness that is substantially equal to that length.

The porous membrane can have the characteristics discussed above. For example, at least one cross-sectional dimension, and preferably a maximum cross-sectional dimension, of the pores can be less than the maximum diameter of the cells. For example, at least one cross-sectional dimension, and preferably the maximum cross-sectional dimension, of the pores can be less than about 40 microns, or less than about 30 microns, or less than about 20 microns, or less than about 15 microns, or less than about 10 microns, e.g., in a range of about 5 microns to about 8 microns. Further, in some embodiments, the membrane can exhibit a surface pore density in a range of about 1×10⁵ to about 2×10⁶ pores/cm². As noted above, a variety of cell types and agents, such as those listed above, can be employed. Further, as noted above, in many embodiments, the cells and the agent(s) to be internalized by the cells can be entrained in a liquid carrier, which can be pushed through the membrane via application of a pressure, e.g., a pressure greater than about 5 psi.

In some embodiments, the transfection of the cells can be achieved at a transfection rate of at least about 40%, or at least about 50%, or at least about 60%, or at least about 70%, or at least about 80%, or at least about 90%. Further, in some embodiments, the cells can be transfected with one or more agents, and the resulting cells have cell viability of greater than about 50%, or greater than about 60%, or greater than about 70%, or greater than about 80%, or greater than about 90%. Cell viability can be measured, for example, using a Beckman Coulter Vi-Cell® counter.

In another aspect, a system for cell processing is disclosed, which includes an inlet port for receiving a fluid carrier containing a plurality of cells and at least one biological agent to be internalized by the cells, a porous membrane in communication with the inlet port for receiving the fluid carrier, said porous membrane having a plurality of pores with at least one cross-sectional dimension less than about 40 microns such that passage of the cells through the pores causes a change in the cells sufficient for entry of the agent into at least a portion of the cells. The system further includes an outlet port that is in communication with said membrane to receive the transfected cells. In some embodiments, the at least one cross-sectional dimension of the pores, and preferably a maximum cross-sectional dimension of the pores, is less than about 40 microns, or less than about 30 microns, or less than about 20 microns, or less than about 15 microns, or less than about 10 microns, e.g., in a range of about 5 microns to about 8 microns. For example, in a such a system, at least one cross-sectional dimension of the pores, and preferably a maximum cross-sectional dimension of the pores, can be in a range of about 2 microns to about 40 microns, or in a range of about 2 microns to about 30 microns, or in a range of about 2 microns to about 20 microns, or in a range of about 2 microns to about 20 microns, or in a range of about 2 microns to about 15 microns, or in a range of about 2 microns to about 10 microns. In some embodiments, the pores of the porous membrane can have a passage length in a range of about 5 microns to about 30 microns.

In some embodiments, the system further includes a mechanism for applying a pressure to the liquid carrier to cause its flow through the porous membrane. By way of example, the mechanism can be include a piston coupled to a syringe in which the liquid carrier is disposed, a pump transferring the liquid carrier from a reservoir to the porous membrane, or any other suitable mechanism. In some embodiments, the pressure mechanism can apply a pressure greater than about 5 psi to the liquid carrier so as to cause its flow through the porous membrane, e.g., at a flow rate greater than about 10 mL/min, e.g., in a range of about 10 to about 20 ml/min, through each pore.

The porous membrane can be formed of a variety of different materials, such as, a metal, a semiconductor, or a polymer. By way of example, in some embodiments, the porous membrane can be formed of a polymeric material, such as polycarbonate, polytetrafluoroethylene (PTFE), polystyrene, polyvinylidene fluoride (PVDF), polyethylene terephthalate (PET), poly methyl methacrylate (PMMA), polypropylene (PP), polyimide (PI), cyclic olefin copolymer (COC), cyclo olefin polymer (COP), polyester, and polydimethylsiloxane (PDMS). In some such embodiments, the pores can be formed in the polymeric material via any of ion track etching, laser drilling, plasma etching, or photolithography.

In some embodiments, the system can further include a porous support that is disposed adjacent to the porous membrane to provide mechanical strength to the membrane so that the membrane can withstand the pressure associated with the flow of a sample therethrough. The porous support can be made of a variety of different materials. In some embodiments, the porous support is a mesh formed of a metal, e.g., stainless steel. In some embodiments, the mesh has openings having a size (e.g., a diameter) greater than about 1 mm.

In some embodiments, the system can further include an input chamber that is disposed upstream of the porous membrane and is in communication with the inlet port for receiving the liquid carrier in which the cells and one or more agents are contained. The input chamber can include an outlet port through which the liquid carrier is introduced onto the porous membrane. In some embodiments, the input chamber can have a volume in a range of about 50 μL to about 1 L. The system can also include a storage reservoir for storing the liquid carrier. The storage reservoir can be in fluid communication with the input chamber for delivering the liquid carrier thereto. Further, an output chamber can be disposed downstream of the porous membrane for collecting the transfected cells.

In a related aspect, a system for parallel cell processing is disclosed, which includes

an inlet support element having a plurality of openings for receiving a plurality of samples containing a fluid carrier, a plurality of cells and at least one agent to be internalized by the cells. The system can further include an outlet support element having a plurality of openings, and a plurality of porous membranes disposed between the inlet support element and the outlet support element and each having a plurality of pores with at least one cross-sectional dimension, and preferably a maximum cross-sectional dimension, less than about 40 microns. Each of the porous membranes can be positioned relative to one of the openings in the inlet support element to receive one of the samples and can be positioned relative to one of the openings in the outlet support element to allow at least a portion of the sample passing therethrough to reach the outlet opening. A pressure applicator coupled to the input support element can be used to apply a pressure, e.g., a pressure greater than about 5 psi, to a sample to push the sample into the inlet ports of the input support element.

In some embodiments, the system can further include a plurality of porous supports (meshes) each disposed adjacent one of said porous membranes to provide mechanical strength thereto. The porous support can be formed, e.g., by a metal mesh, such as a stainless steel mesh.

In some embodiments, the porous membranes can include a polymeric material, such as those discussed above. Further, as noted above, in some embodiments, the pores can be formed in such a polymeric material via any of ion track etching, laser drilling, plasma etching, or photolithography. Further, the pores of the porous membrane can exhibit a substantially uniform, or a non-uniform, cross-sectional dimension along their lengths. The pores of the porous membrane can have inlet ports that are substantially flush with a top surface of the membrane and outlet ports that are substantially flush with the bottom surface of the membrane.

In a related aspect, a method of delivering one or more gene editing system(s) and/or component(s) to a cell is disclosed, which includes passing at least one cell through a pore of a membrane having a plurality of pores each having at least one cross-sectional dimension, and preferably a maximum cross-sectional dimension, less than a diameter of the cell, and exposing the cell to at least gene editing component and/or system, e.g., as described herein, as the cell passes through the pore, wherein the cell undergoes a change as it passes through the pore sufficient to allow the cell to uptake said at least one gene editing component and/or system. For example, at least one cross-sectional dimension, and preferably a maximum cross-sectional dimension, of the pores can be less than about 40 microns (e.g., in a range of about 2 microns to about 40 microns), or less than about 30 microns (e.g., in a range of about 2 microns to about 30 microns), or less than about 20 microns (e.g., in a range of about 2 microns to about 20 microns), or less than about 15 microns (e.g., in a range of about 2 microns to about 15 microns), or less than about 10 microns (e.g., in a range of about 2 microns to about 10 microns), e.g., in a range of about 5 microns to about 8 microns. In some embodiments of the above gene editing method, the membrane has a surface pore density in a range of about 1×10⁵ to about 2×10⁶ pores/cm². Further, in some embodiments, the membrane can be at least partially coated with a polymeric material, such as polyvinylpyrrolidone.

In some embodiments, the uptake of the one or more molecules (e.g., one or more genetic components and/or one or more components of a gene editing system) results in modulation of the expression of a gene, e.g., a transgene or target gene, by at least about any of 15%, 20%, 30%, 40%, 50%, 60%, 70%, 80%, 90%, 100%, 120%, 130%, 140%, 150%, 160%, 170%, 180%, 190%, 200%, 210%, 220%, 230%, 240%, and 250%. In some embodiments, the modulation is reduced expression. In other embodiments, the modulation is enhanced expression.

A variety of different gene editing systems and components can be used, for example, as described herein. In many embodiments, the cell's membrane is normally impermeable to the gene editing component(s) and/or system(s) that are delivered to the cell via the above methods. In some embodiments, the gene editing component(s) and/or system(s) can include any of a protein, a deoxyribonucleic acid (DNA), a ribonucleic acid (RNA), a peptide, a lipid, a non-permeable compound, a plasmid, and a ribonucleoprotein complex (RNP), e.g., a CRISPR gene editing system comprising one or more RNPs, e.g., as described herein, and combinations thereof. By way of example, the RNP can be a Cas9-gRNA complex. In some embodiments, the change induced in the cell to facilitate the uptake of the gene editing component(s) and/or system(s) can be a physical and/or a chemical change. By way of example, such a change can be a change in the permeability of the cell's membrane. In some embodiments, the change can be transient.

In some embodiments of the gene editing methods described herein, including the above gene editing method, the cell and the at least one gene editing component and/or system can be disposed within a liquid carrier and the liquid carrier is pushed through the pore via application of a pressure thereto. In some cases, the applied pressure can cause a flow of the liquid carrier through each pore of the membrane at a flow rate of at least about 10 ml/min, e.g., in a range of about 10 to about 20 ml/min.

The gene editing methods described herein can be used to deliver gene editing components and/or systems to a variety of different cells. For example, the cell can be any of a progenitor cell, an immune effector cell (e.g., a T cell or NK cell), a human embryonic stem cell (hES cell), an induced pluripotent stem cell (iPSC), a mesenchymal stem cell, a keratinocyte, and a human bronchial epithelial cell. In some embodiments the cell is a T cell. In other embodiments the cell is a hematopoietic stem and progenitor cell (HSPC), e.g., a CD34+ cell.

In some embodiments, the gene editing methods described herein, including the above gene editing method, can further include the step of selecting one or more cells from a collection of heterogeneous cells followed by passing those cells through the porous membrane while exposing the cells to one or more gene editing agents so as to cause the delivery of those gene editing agent(s) into the cells. A variety of selection criteria can be used. For example, in some embodiments, one or more cell markers, such as a CD3, CD4, CD8, CD27, CD28, CD34, CD90, CD49f and combinations thereof, can be used to select the cells. In some embodiments, the selection of the cells can be based on their size.

In a related aspect, a system for cell processing is disclosed, which comprises an inlet port for receiving a plurality of cells, and a plurality of porous membranes positioned in series relative to one another for sequentially receiving at least a portion of said cells via said inlet port, wherein each of said porous membranes has at least one cross-sectional dimension less than about 40 microns, e.g., in a range of about 2 microns to about 40 microns, or in a range of about 2 microns to about 30 microns, or in a range of about 2 microns to about 20 microns, or in a range of about 2 microns to about 15 microns. In some embodiments, each of the porous membranes has a maximum cross-sectional dimension less than about 40 microns. In some embodiments, the system can further include a plurality of liquid delivery modules each configured to exposed the cells to an agent such that at least some of the cells uptake the agent as they pass through one of the porous membranes. In some such embodiments, the liquid delivery modules are configured to expose the cells to different agents as the cells pass through different ones of the porous membranes.

Further understanding of various aspects of the invention can be obtained by reference to the following detailed description in conjunction with the associated drawings, which are described below. The drawings are not necessarily drawn to scale.

### BRIEF DESCRIPTION OF THE DRAWINGS

FIG. 1 is a flow chart depicting a step of a method according to an embodiment of the present teachings for delivering agent(s) into cells,

FIG. 2 is a flow chart depicting the steps of a method according to another embodiment of the present teachings for selecting cells from a collection of heterogeneous cells and delivering one or more agent(s) to the selected cells,

FIG. 3A is a flow chart depicting the steps of a method according to an embodiment of the present teachings for gene editing,

FIG. 3B schematically depicts an example of various steps of a cycle in an exemplary clinical use of gene editing techniques according to the present teachings,

FIG. 3C schematically depicts another example of various procedures of a cycle in an exemplary clinical use of gene editing techniques according to the present teachings,

FIG. 4A schematically depicts a system according to an embodiment of the present teachings for processing cells, e.g., to deliver agents into cells,

FIG. 4B is a schematic partial view of the system depicted in FIG. 4A illustrating a plurality of wells for collecting the processed cells,

FIG. 5A schematically depicts a cell processing assembly employed in the system depicted in FIG. 4A,

FIG. 5B is an exploded view of the cell processing assembly depicted in FIG. 5A and a fitting, a tubing and a needle for fluidly coupling the cell processing assembly to a syringe containing a cell sample,

FIG. 5C is a schematic cross-sectional view of the cell processing assembly coupled to a syringe,

FIG. 5D is another schematic cross-sectional view of the cell processing assembly,

FIG. 6A is a schematic view of an input block of the cell processing assembly,

FIG. 6B is a schematic view of an output block of the cell processing assembly,

FIG. 7 is a schematic view of an embodiment of a porous membrane for use in a cell processing system according to the present teachings (only a few pores are depicted for ease of illustration),

FIG. 8 is a schematic view of another embodiment of a porous membrane having pores with a rectangular cross-sectional shape,

FIG. 9 schematically depicts a pore of a porous membrane having non-uniform cross-sectional dimension along its length,

FIG. 10 schematically depicts a mesh that can be disposed adjacent a porous membrane in a cell processing system

FIG. 11A schematically depicts another embodiment of a cell processing system according to the present teachings,

FIG. 11B is a schematic exploded view of the cell processing system depicted in FIG. 11A,

FIG. 11C is a partial schematic cross-sectional view of the system depicted in FIGS. 11A and 11B,

FIG. 12A schematically depicts a system according to an embodiment that includes a cell selection module and a cell processing module in communication with the cell selection module,

FIG. 12B schematically depicts an exemplary cell sorting device employing magnetic forces for separating cells,

FIG. 13A schematically depicts a system according to an embodiment of the present teachings, which includes a microfluidic device for delivering agents to cells,

FIG. 13B schematically depicts a system according to another embodiment of the present teachings, which includes a plurality of porous membranes positioned in series relative to one another,

FIG. 14 depicts delivery efficiency of a prototype device according to an embodiment of the present teachings demonstrated via indel % through NGS, B2M KD by FACS analysis and cell recovery through Vi-Cell of varying cell density per 50 μL of delivery volume with RNP concentration of 200 μg of Cas9/50 μg sg RNA,

FIG. 15 depicts indel formation and B2M knockdown data illustrating the scalability of a prototype device according to an embodiment of the present teachings for a cell density up to 10 million cells per 50 μL with RNP concentration of 200 μg of Cas9/50 μg sgRNA,

FIG. 16 depicts dose response of RNP complex at 50 μg or 200 μg Cas9 with 5 million cells per 50

FIG. 17 depicts delivery efficiency via indel % through NGS, B2M KD via FACS analysis and cell recovery through Vi-Cell of membranes per 50 μL of delivery volume with 5 million cells and RNP concentration of 200 μg of Cas9/50 μg sgRNA using a prototype device according to an embodiment of the present teachings,

FIG. 18 depicts data indicating editing and cell recovery rates for delivery of Cas9/RNP complex at 200 μs of Cas9 into human hematopoietic stem cells at a concentration of 5 million cells per 50 μL as a function of applied pressure for passing the cells through a porous membrane in accordance with an embodiment of the present teachings,

FIG. 19A depicts HSC surface markers by flow cytometry after 7 day expansion of 50 k/mL seeded cells 2 days after mock treatment via TRIAMF or neon electroporation. Comparison of CD34 and CD90 percentages by flow cytometry staining are also shown,

FIG. 19B depicts HSC cell count after 7 day expansion of 50 k/mL seeded cells 2 days after mock treatment of TRIAMF or neon electroporation. Comparison of total, CD34+, and Cd34+/CD90+ cell counts via ViCell with flow cytometry staining is also depicted,

FIG. 19C depicts HSC fold expansion after 7 day expansion of 50 k/mL seeded cells 2 days after mock treatment via TRIAMF or neon electroporation,

FIG. 20 depicts percentage of HbF cells at D7 and D14 of erythroid differentiation protocol of WT, mock treated, HBG edited, and UNC0638 positive control treated cells. Cells were analyzed by fixation and staining of CD235a, CD71, and HbF,

FIG. 21A depicts HSC surface markers by flow cytometry after 7 day expansion of 50 k/mL seeded cells 2 days after recovery of WT cells, mock TRIAMF treated cells, and HBG edited cells via TRIAMF. Comparison of CD34 and CD90 percentages by flow cytometry staining is also shown,

FIG. 21B depicts HSC cell count after 7 day expansion of 50 k/mL seeded cells 2 days after recovery of WT cells, mock TRIAMF treated cells, and HBG edited cell via TRIAMF. Comparison of total, CD34+, and Cd34+/CD90+ cell counts via ViCell with flow cytometry staining is also shown,

FIG. 21C depicts HSC fold expansion after 7 day expansion of 50 k/mL seeded cells 2 days after recovery of WT cells, mock TRIAMF treated cells, and HBG edited cell via TRIAMF,

FIG. 22 depicts the result of colony forming assay to determine the TRIAMF treated HSC's ability to commit to different lineages. Colony counts of different haematapoeitic lineages after 14 days of growth on methocult are shown (plates were imaged and analyzed with StemVision),

FIG. 23 presents data indicating that TRIAMF edited HSPCs display long-term reconstitution in NSG mice. 700 k TRIAMF treated cells were tail vein injected into each mouse (WT: N=4, Mock: N=5, HBG: N=6). Peripheral blood was collected every 4 weeks and stained for anti human and anti mouse CD45 and human CD45,

FIG. 24 depicts the results of flow cytometry analysis of GFP expression in HSCs at different time points after delivery of 60 μg of mRNA in accordance with an embodiment of the present teachings, along with percentage cell recovery after 24 hours,

FIG. 25 depicts the results of flow cytometry analysis of GFP expression in HSCs at different time points after delivery of minicircle DNA at 5, 10 and 20 μg of DNA in accordance with an embodiment of the present teachings, along with percentage cell recovery after 24 hours, and

FIG. 26 depicts the results of flow cytometry analysis of Cy5 signal in HSCs at different time points after delivery of 30 μg of CY5-tagged peptides, along with percentage cell recovery after 24 hours.

### DETAILED DESCRIPTION

The present teachings are generally directed to methods and systems for cell processing, and more particularly, to methods and systems that cause a change in a cell (e.g., a transient physical deformation) that in turn allows the uptake of chemical and/or biological compounds, including those that cannot be normally internalized by the cell, e.g., due to impermeability of the cell's membrane. In one aspect, a porous membrane, e.g., a porous polymeric membrane, having a plurality of pores with a maximum cross-sectional dimension less than the diameter of a cell undergoing processing is employed to cause a change in the cell suitable for mediating the uptake of an agent by the cell. In certain embodiments, the membrane has two essentially planar opposing surfaces. Without being limited to any particular theory, the passage of such a cell through such pores can subject to the cell to compressive forces along multiple directions (e.g., along at least two orthogonal directions), which can cause a change in the cell sufficient to allow the cell to uptake an agent to which the cell is concurrently exposed.

Various terms are used herein consistent with their meanings as understood by those having ordinary skill in the art. By way of further illustration, several terms are defined as follows.

The term “cell” is used herein consistent with its ordinary meaning to refer to the smallest structural and functional unit of an organism. Although in many of the embodiments the teachings of the invention are discussed in connection with their application to mammalian cells, the present teachings are broadly applicable to any prokaryotic or eukaryotic cell.

The term “diameter of a cell” or “a cell's diameter” as used herein refers to a maximum cross-sectional dimension of a cell, which can have a variety of different shapes depending, e.g., on the cell type.

The term “cell viability” refers generally to a measure of the proportion of live cells in a population of cells. Examples of methods used to measure cell viability include dead cell stains such as Sytox® or Trypan blue staining. In some embodiments, cell viability is measured on a Beckman Coulter Vi-Cell® counter according to standard protocols. The term “cell recovery” generally refers to a measure of the proportion of live cells of the treated group relative to an untreated group as counted by Vi-Cell 24-48 hours after treatment.

The term “membrane” refers to a structure, which in many embodiments is essentially planar, having a thickness that is significantly less than a transverse (e.g., width) thereof. For example, in some embodiments, the thickness of the membrane can be 100 times more or less than a diameter of the membrane.

The term “essentially planar” as used herein refers to structures having opposing surfaces, e.g., an inlet surface and an outlet surface that are parallel to each other or are within 10 degrees, or 5 degrees, or 3 degrees, or 1 degree of being parallel to each other.

The term “transfection” is used herein broadly to refer to a process of introducing an agent, e.g., a ribonucleic acid protein complex, into a cell.

The term “transfection efficiency” as used herein refers to the fraction of cells passing through a porous membrane while exposed to at least one agent according to the present teachings that have internalized that agent.

The term “agent” as used herein refers to any inorganic or organic molecule, compound, and molecular complex, biological organism and combinations thereof. Non-limiting examples of agents are provided below.

The term “about” as applied to a numerical value or a numerical range as used herein denotes a plus or minus variation of at most 10% of the numerical value or the range.

The term “substantially” as used herein denotes a deviation of at most 5% relative to a complete condition or state.

The term “cell membrane” or “membrane of a cell” are used interchangeably to refer to a semipermeable membrane that separates the interior of a cell from the environment external to the cell.

The term “protein complex” as used herein refers to a protein and at least one binding partner that collectively form a complex. By way of example, the binding partner can be one or more proteins, one or more nucleic acids, a combination of one or more proteins and one or more nucleic acids. The binding of a protein to a binding partner in a protein complex can be achieved via covalent or non-covalent interactions. For example, a protein complex can be a protein-nucleic acid complex, such as a RNA/protein ribonuclear protein complex (RNP).

The term “circulatory cell” and its plural “circulatory cells” are used herein to refer to non-adherent cells, such as hematopoietic cells, e.g., hematopoietic stem and progenitor cells, stem cells found in bone marrow, cord blood, peripheral blood as well as immune effector cells, such as T cells.

The term “active surface area” of the membrane as used herein refers to a surface area of the membrane that is exposed to a liquid carrier containing cells and agent(s) of interest.

The term “a viability enhancer,” as used herein refers to a compound which causes cells, e.g., HSPCs, HSCs and/or HPCs to proliferate, e.g., increase in number, at a faster rate relative to the same cell types absent said compound. In one exemplary aspect, the cell type is a stem cell, for example, an HSPC, and the viability enhancer is an inhibitor of the aryl hydrocarbon receptor pathway. In some embodiments, the proliferation, e.g., increase in number, is accomplished ex vivo. Additional examples of viability enhancers are described in, for example, WO2010/059401 (e.g., the molecule described in Example 1 of WO2010/059401), the contents of which is incorporated herein in its entirety, and are also described in, for example, WO2013/110198, the contents of which is incorporated herein in its entirety. In some embodiments, the viability enhancer is ((S)-2-(6-(2-(1H-indol, yl)ethylamino-2-(5-fluoropyridin-3-yl)-9H-purin-9-yl)propan-1-ol; compound 157S according to WO2010/059401. In some embodiments, the viability enhancer is 4-[2-[[2-(1-benzothiophen-3-yl)-9-propan-2-ylpurin-6-yl]amino]ethyl]phenol); compound 1 according to WO2010/059401 (also referred to as StemRegenin 1 or SR1). In some embodiments, the viability enhancer is UM171. In some embodiments, the viability enhancer comprises a combination of two or more agents, for example, comprises a combination of UM171 and an aryl hydrocarbon receptor inhibitor, for example, SR1.

As used herein in connection with a gene editing system, the term “template nucleic acid” refers to one or more nucleic acid molecules comprising sequence which can be inserted at a target sequence (for example, a sequence cut by a gene editing system). Without being bound by theory, it is believed that such insertion can be accomplished by homology-directed repair (HDR). In some embodiments, the template nucleic acid is a single strand oligonucleotide, for example a single strand DNA molecule or single strand RNA molecule. In some embodiments, the template nucleic acid is a double strand nucleic acid, for example a double strand DNA molecule, for example a plasmid. In some embodiments, the template nucleic acid is present on a vector, for example, an adeno-associated viral (AAV) vector. In some embodiments, upon insertion of nucleic acid sequence from the template nucleic acid, a target mutation in a genome is corrected. In some embodiments, upon insertion of nucleic acid sequence from the template nucleic acid, a heterologous gene or expression cassette is inserted into the genome.

The present methods and systems can be particularly effective for transfecting circulatory cells, and in particular hematopoietic stem cells (HSCs), when certain parameters of the methods and/or the systems are adjusted in a manner discussed herein. For example, at least one of the membrane pore size, including maximum cross-sectional dimension and/or the length of the pores, the concentrations of the cells and/or transfecting agent(s) can be selected as discussed in more detail herein so as to greatly enhance both transfection rate and cell viability following transfection. In some embodiments, in addition to the above parameters, the active surface area of the membrane and/or the pore density of the membrane are selected in a manner discussed herein to enhance the transfection rate and cell viability. In some embodiments, multiple of the above parameters are selected to be within the ranges discussed herein to ensure unexpectedly high transfection rate and cell viability, particularly for the transfection of hematopoietic stem cells.

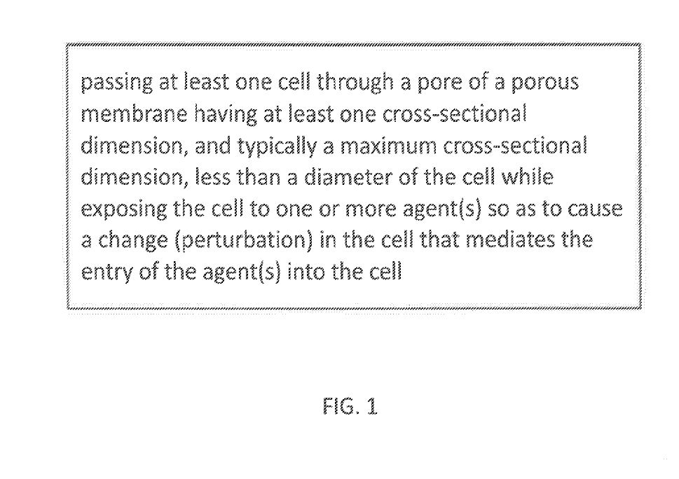

FIG. 1 is a flow chart depicting a step of a method according to an embodiment of the present teachings for delivering agent(s) into cells. With reference to the flow chart of FIG. 1, In some embodiments of a method according to the present teachings for processing cells, at least one cell is passed through a pore of a porous membrane having at least one cross-sectional dimension less than a diameter of the cell while exposing the cell to one or more agents so as to cause a change (a perturbation) in the cell that mediates entry of the agent(s) into the cell. In some embodiments, each pore of the porous membrane can have at least one cross-sectional dimension, and preferably a maximum cross-sectional dimension, less than the cell's diameter. As discussed in more detail below, the use of a porous membrane can allow concurrent processing of a plurality of cells, thus significantly increasing the throughput compared to conventional methods.

The cellular change caused by the passage of the cell through a pore of the porous membrane can be any of a physical and/or chemical change suitable for mediating the internalization of the agent(s) by the cell. For example, and without being limited to any particular theory, the passage of a cell through a pore having a maximum cross-sectional dimension less than the cell's dimeter can subject the cell to compressive forces exerted by the pore wall. Such compressive forces can cause physical deformation of the cell, which can in turn mediate the uptake of the agent(s) by the cell. For example, the physical deformation of the cell can change the permeability of the cell's membrane to the agent(s), e.g., via generation of transient pores in the membrane, thereby allowing the cell to internalize the agent(s).

In some embodiments, at least one cross-sectional dimension (and preferably the maximum cross-sectional dimension) of the pores can be in a range of about 2 microns to about 40 microns, or in a range of about 2 microns to about 30 microns, or in a range of about 2 microns to about 20 microns, or in a range of about 2 microns to about 15 microns, or in a range of about 2 microns to about 10 microns, e.g., in a range of about 5 microns to about 8 microns. Further, in some embodiments, the passage length of the pores can be in a range of about 5 microns to about 30 microns, e.g., in a range of about 7 microns to about 21 microns, though other lengths can also be utilized. In some embodiments, the porous membrane can have a surface pore density in a range of about 1×10⁵ to about 1×10⁶ pores/cm². In some embodiment, the porous membrane can be formed of a suitable polymeric material, such as those discussed above.

The pores of the porous membrane can have hydrophilic or hydrophobic inner surfaces. In some cases, the porous membrane, and particularly the inner surfaces of the pores, can be coated with one or more compounds. Such coating of the surfaces can be performed for a variety of reasons. For example, the porous membrane can be coated to enhance the smoothness of the inner surfaces of the pores and/or to enhance the degree of hydrophobicity or hydrophilicity of those surfaces. By way of example, in one embodiment, the porous membrane can be coated with a thin film (e.g., a film having a thickness in a range of about several nanometers to several millimeters) of polyvinylpyrrolidone. Other suitable coating materials can include, for example, polyethylene glycol (PEG) and PEG-based compounds, polyvinyl alcohol (PVA), hydroxyethylcellulose, tween 20, octadecylsilane, Brij35, poly(p-xylenes), fluoropolymers, Pluronic F127.

In some embodiments, the pores of the membrane can be substantially homogenous. For example, in some embodiments, all the pores of the membrane can have substantially uniform diameters (i.e., different pores can have substantially identical diameters). Further, in some embodiments, the diameter of any given pore can be substantially uniform along its entire length, i.e., the pore can exhibit substantially the same diameter across its entire length. In other words, at least some pores, and in some embodiments all pores, do not exhibit any constriction zone. In some embodiments, all the pores have uniform diameters along their entire lengths and the diameters of different pores are substantially identical.

The above method of cell processing can be applied to different cell types. Some examples of suitable cell types include, without limitation, a progenitor cell, an immune effector cell (e.g., a T cell or NK cell), a human embryonic stem cell (hES cell), an induced pluripotent stem cell (iPS), a mesenchymal stem cell, a keratinocyte and a human bronchial epithelial cell, among others. In some embodiments, the cell can be a hematopoietic stem and progenitor cell (HSPC), e.g., a CD34+ cell. In some embodiments, the cell can be a T cell, e.g., a CD4+ T cell and/or CD8+ T cell.

Further, a variety of different biological and non-biological agents (e.g., molecules and/or complexes), including organic and inorganic compounds, can be introduced into a cell using the methods of the present teachings. In many embodiments, the cell's membrane can be normally impermeable to the agent. The agent can be neutral or electrically charged. Further, the agent can have a variety of different molecular weights.

Some examples of agents that can be internalized by a cell using the present teachings include, without limitation, a deoxyribonucleic acid (DNA), a ribonucleic acid (RNA), a plasmid, a ribonuclear protein complex (RNP), a protein, a peptide, a lipid, a non-membrane permeable compound, and a dye. For example, the agent can be a Cas9-gRNA RNP complex, e.g., as described herein. By way of example, in some embodiments, the methods and apparatus of the present teachings can be used to modify an immune effector cell, e.g., a T cell, by introducing a gene editing system, e.g., a CRISPR gene editing system, e.g., a Cas9-gRNA RNP complex into the cell so as to selectively modify the T cell's genome, e.g., to reduce or eliminate expression of an endogenous gene in order to, for example, reduce or eliminate the presence of a protein, e.g., a surface protein, in said cell and/or its progeny. By way of a further example, in some embodiments, the methods and apparatus of the present teachings can be used to modify an HSPC, by introducing a gene editing system, e.g., a CRISPR gene editing system, e.g., a Cas9-gRNA RNP complex into the cell so as to selectively modify the HSPC's genome, e.g., to reduce or eliminate expression of an endogenous gene in order to, for example, reduce or eliminate the presence of a protein, e.g., a surface protein, in said cell and/or its progeny. In some embodiments, more than one gene editing system, e.g., CRISPR gene editing system, e.g., Cas9-gRNA RNP complex, can be introduced simultaneously or sequentially into said cells using the methods and apparatus described herein, to, for example, selectively modify more than one gene within the cell's genome, or selectively modify more than one site within a single gene. Gene editing systems, e.g., CRISPR gene editing systems, useful in such methods are described herein in additional detail below.

In some embodiments, a population of cells can be passed through a system according to the present teachings multiple times in series, with the optional step of resting the cells between each passage. For example, the cells can be collected after passage through the system, and reintroduced to the system to pass through the porous membrane again. This process can be repeated as needed. In some such embodiments, the agent(s) and/or the medium (carrier liquid) to which the cells are exposed can be optionally changed in different passes of the cells through the system, e.g., to introduce different molecules/complexes to the cells. For example, a sample of cells can be exposed to a solution containing a molecule/complex during a first pass through the system so as to deliver that molecule/complex to the cells. The transfected cells can be passed through the system a second time while exposing them to a different solution containing a different molecule/complex so as to deliver the second molecule/complex to the cells. This process can be repeated as many times as needed.

In some embodiments, the porous membrane can be used to transfect a plurality of cells with an agent, e.g., a biological agent to which the cell's membrane is normally impermeable, with a transfection rate of at least about 40%, or at least about 50%, or at least about 60%, or at least about 70%, or at least about 80%, or at least about 90%. Further, in some embodiments, such transfection can be performed while achieving a cell viability of greater than about 50%, or greater than about 60%, or greater than about 70%, or greater than about 80%, or greater than about 90%.

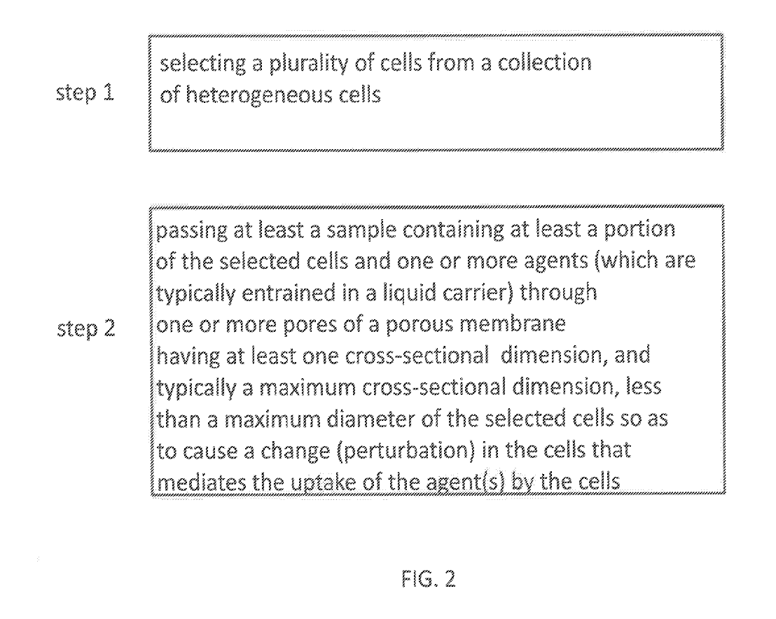

FIG. 2 is a flow chart depicting the steps of a method according to another embodiment of the present teachings for selecting cells from a collection of heterogeneous cells and delivering one or more agent(s) to the selected cells. With reference to the flow chart of FIG. 2, in a related aspect, a method of processing cells is disclosed, which includes selecting a plurality of cells from a collection of heterogeneous cells (step 1). The selection can be achieved based on a variety of different criteria. For example, in some cases, a plurality of cells that preferentially express certain surface proteins can be selected for further processing. For example, the selected cells can have any of CD3, CD4, CD8, CD27, CD28, CD34, CD90, CD49f, or combinations thereof. Such selection can be done, for example, by utilizing a solid support, such as a magnetic bead, conjugated to an antibody which binds the marker or markers of interest. In some cases, a subset of the cells having a diameter in a certain range can be selected for subsequent processing. In some embodiments, the selected cells can be HSPCs and can be selected based on expression of, for example, CD34, alone or in combination with another marker or markers, e.g., CD90. In some embodiments, the selected cells can be T cells, and can be selected based on expression of, for example, CD3, CD4 and/or CD8, alone or in combination with another marker or markers.

A sample containing the selected cells and one or more agents to be internalized by the cells, which are typically entrained in a liquid carrier, can be passed through a porous membrane having a plurality pores, each of which has at least one cross-sectional dimension (and preferably a maximum cross-sectional dimension) less than a maximum diameter of the cells, such that the passage of the cells through the pores causes a change (e.g., a physical deformation) of the cells sufficient for the uptake of the agent(s) by the cells (step 2). The porous membrane can have the properties discussed above. A plurality of cells and agents, such as those discussed above, can be employed in such a method.

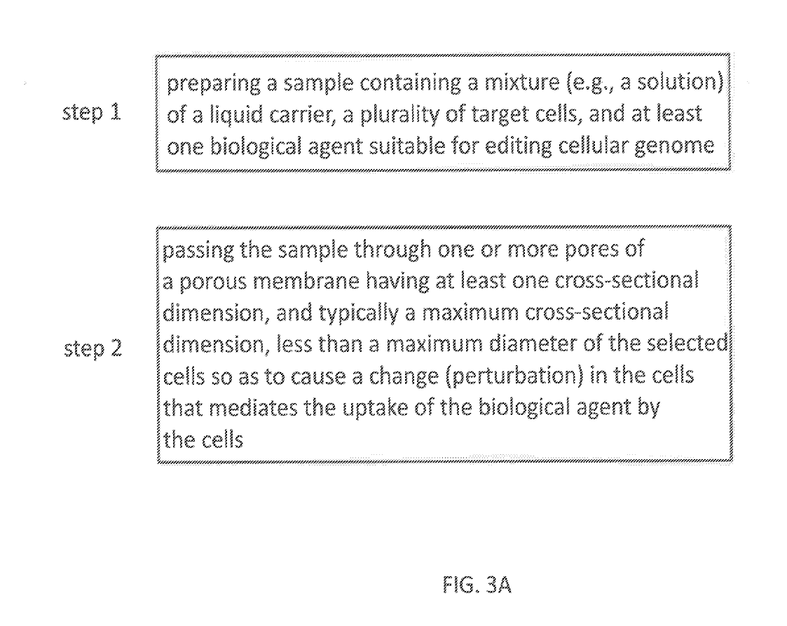

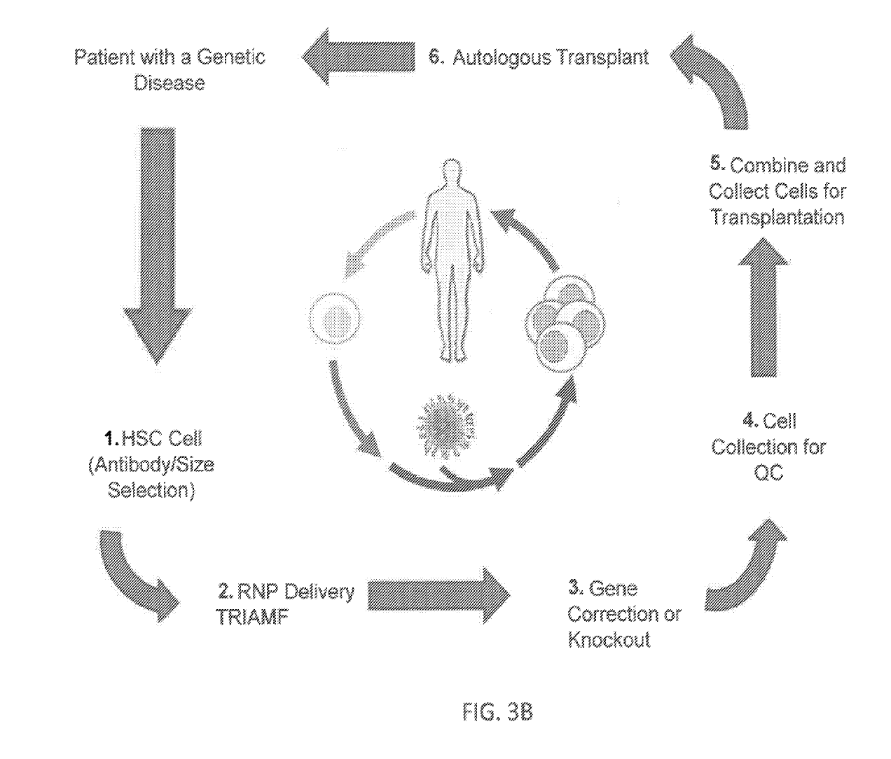

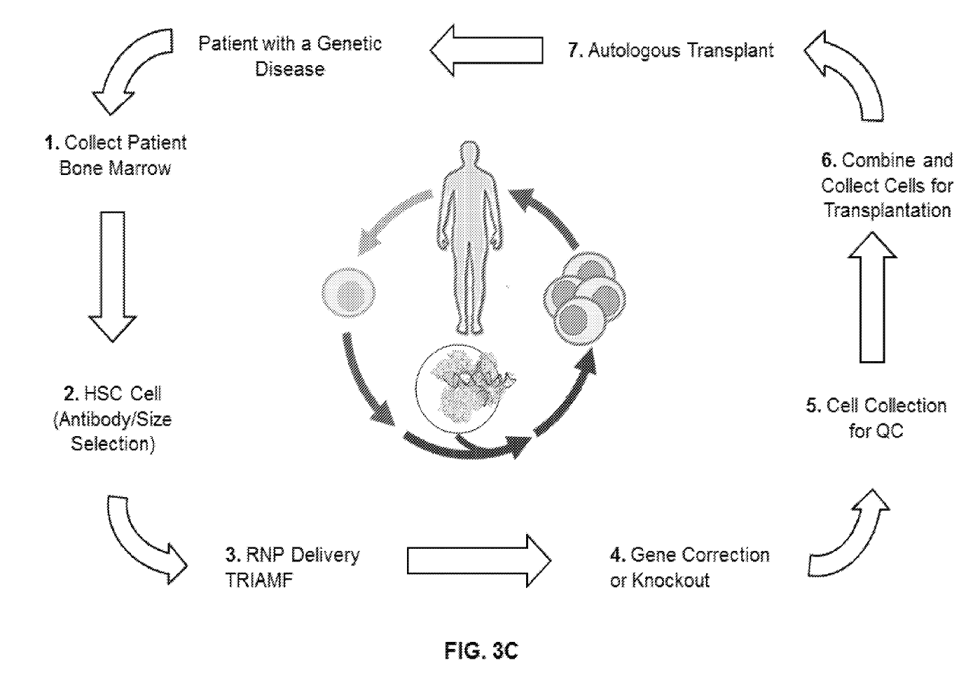

With reference to the flow chart of FIG. 3A, a method of editing the genome of cells is disclosed, which includes preparing a mixture (e.g., a solution) of a liquid carrier, a plurality of target cells and at least one biological agent suitable for editing cellular genome (step 1), and passing the mixture through pores of a porous membrane, each of which has at least one cross-sectional dimension less than a maximum diameter of the cells, so as to cause a change (e.g., a physical and/or chemical modulation) in the cells that would allow the cells to uptake the biological agent(s). The uptake of the biological agent by a cell can lead to selective editing of its cellular genome. For example, the internalized biological agent can mediate selective insertion of DNA segment(s) into, or deletion of DNA segments (e.g., one or more nucleotides), from the genome. In some embodiments, the above method can allow performing such insertion/deletion editing at an efficiency of greater than about 20%, e.g., greater than about 50%, 60%, 70%, 80%, 90%, 95% or more. In other words, of all the cells passing through the membrane, at least 20%, e.g., at least about 50%, 60%, 70%, 80%, 90%, 95% or more, of those cells would undergo a genomic edit, e.g., as measured by next generation sequencing of the target locus or loci. By way of example, in some embodiments, the gene editing methods according to the present teachings can be employed to modulate the expression of a target gene by at least about 15%, or at least about 20%, or 25%, or 30%, or 40%, or 50%, or 60%, or 70%, or 80%, or 90%, or 100%, or 110%, or 120%, or 130%, or 140%, or 150%, or 160%, or 170%, or 180%, or 190%, or 200%, or 210%, or 220%, or 230%, or 240%, or 250%. In some embodiments, the modulation can be reduced expression. In other embodiments, the modulation can be enhanced expression.

In some embodiments, the gene editing component can be a protein-nucleic acid complex. By way of example, such a protein-nucleic acid complex can include a Cas protein and a guide RNA (gRNA). For example, the protein-nucleic acid complex can be a complex of a Cas9 protein and a gRNA. In some embodiments, the protein can be, for example, a Zinc-finger nuclease (ZFN), a transcription activator-like effector nuclease (TALEN), a mega nuclease, or Cre recombinase, among others.

### Gene Editing Systems

According to the present invention, gene editing systems or one or more components of a gene editing system can be delivered using the methods and apparatus described herein. As used herein, the term “gene editing system” refers to a system, e.g., one or more molecules or molecular complexes, that direct and effect an alteration or modification, e.g., an insertion or deletion, of one or more nucleic acids at or near a site of genomic DNA targeted by said system. Exemplary gene editing systems are known in the art, and are described more fully below.

### CRISPR Gene Editing Systems

Naturally-occurring CRISPR systems are found in approximately 40% of sequenced eubacteria genomes and 90% of sequenced archaea. Grissa et al. (2007) BMC Bioinformatics 8: 172. This system is a type of prokaryotic immune system that confers resistance to foreign genetic elements such as plasmids and phages and provides a form of acquired immunity. Barrangou et al. (2007) Science 315: 1709-1712; Marragini et al. (2008) Science 322: 1843-1845.

The CRISPR system has been modified for use in gene editing (silencing, enhancing or changing specific genes) in eukaryotes such as mice, primates and humans. Wiedenheft et al. (2012) Nature 482: 331-8. This is accomplished by, for example, introducing into the eukaryotic cell one or more vectors encoding a specifically engineered guide RNA (gRNA) (e.g., a gRNA comprising sequence complementary to sequence of a eukaryotic genome) and one or more appropriate RNA-guided nucleases, e.g., Cas proteins. The RNA guided nuclease forms a complex with the gRNA, which is then directed to the target DNA site by hybridization of the gRNA's sequence to complementary sequence of a eukaryotic genome, where the RNA-guided nuclease then induces a double or single-strand break in the DNA. Insertion or deletion of nucleotides at or near the strand break creates the modified genome.

As these naturally occur in many different types of bacteria, the exact arrangements of the CRISPR and structure, function and number of Cas genes and their product differ somewhat from species to species. Haft et al. (2005) PLoS Comput. Biol. 1: e60; Kunin et al. (2007) Genome Biol. 8: R61; Mojica et al. (2005) J Mol. Evol. 60: 174-182; Bolotin et al. (2005) Microbiol. 151: 2551-2561; Pourcel et al. (2005) Microbiol. 151: 653-663; and Stern et al. (2010) Trends. Genet. 28: 335-340. For example, the Cse (Cas subtype, E. coli) proteins (e.g., CasA) form a functional complex, Cascade, that processes CRISPR RNA transcripts into spacer-repeat units that Cascade retains. Brouns et al. (2008) Science 321: 960-964. In other prokaryotes, Cas6 processes the CRISPR transcript. The CRISPR-based phage inactivation in E. coli requires Cascade and Cas3, but not Cas1 or Cas2. The Cmr (Cas RAMP module) proteins in Pyrococcus furiosus and other prokaryotes form a functional complex with small CRISPR RNAs that recognizes and cleaves complementary target RNAs. A simpler CRISPR system relies on the protein Cas9, which is a nuclease with two active cutting sites, one for each strand of the double helix. Combining Cas9 and modified CRISPR locus RNA can be used in a system for gene editing. Pennisi (2013) Science 341: 833-836.

In some embodiments, the RNA-guided nuclease is a Cas molecule, e.g., a Cas9 molecule. Cas9 molecules of a variety of species can be used in the methods and compositions described herein. While the S. pyogenes Cas9 molecule are the subject of much of the disclosure herein, Cas9 molecules of, derived from, or based on the Cas9 proteins of other species listed herein can be used as well. In other words, other Cas9 molecules, e.g., S. thermophilus, Staphylococcus aureus and/or Neisseria meningitidis Cas9 molecules, can be used in the systems, methods and compositions described herein. Additional Cas9 species include: Acidovorax avenae, Actinobacillus pleuropneumoniae, Actinobacillus succinogenes, Actinobacillus suis, Actinomyces sp., cycliphilus denitrificans, Aminomonas paucivorans, Bacillus cereus, Bacillus smithii, Bacillus thuringiensis, Bacteroides sp., Blastopirellula marina, Bradyrhiz obium sp., Brevibacillus latemsporus, Campylobacter coli, Campylobacter jejuni, Campylobacter lad, Candidatus Puniceispirillum, Clostridiu cellulolyticum, Clostridium perfringens, Corynebacterium accolens, Corynebacterium diphtheria, Corynebacterium matruchotii, Dinoroseobacter sliibae, Eubacterium dolichum, gamma proteobacterium, Gluconacetobacler diazotrophicus, Haemophilus parainfluenzae, Haemophilus sputorum, Helicobacter canadensis, Helicobacter cinaedi, Helicobacter mustelae, Ilyobacler polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica. Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tislrella mobilis, Treponema sp., or Verminephrobacter eiseniae.

A Cas9 molecule, as that term is used herein, refers to a molecule that can interact with a gRNA molecule (e.g., sequence of a domain of a tracr) and, in concert with the gRNA molecule, localize (e.g., target or home) to a site which comprises a target sequence and PAM sequence.

In some embodiments, the ability of an active Cas9 molecule to interact with and cleave a target nucleic acid is PAM sequence dependent. A PAM sequence is a sequence in the target nucleic acid. In some embodiments, cleavage of the target nucleic acid occurs upstream from the PAM sequence. Active Cas9 molecules from different bacterial species can recognize different sequence motifs (e.g., PAM sequences). In some embodiments, an active Cas9 molecule of S. pyogenes recognizes the sequence motif NGG and directs cleavage of a target nucleic acid sequence 1 to 10, e.g., 3 to 5, base pairs upstream from that sequence. See, e.g., Mali et al, SCIENCE 2013; 339(6121): 823-826. In some embodiments, an active Cas9 molecule of S. thermophilus recognizes the sequence motif NGGNG and NNAG AAW (W=A or T) and directs cleavage of a core target nucleic acid sequence 1 to 10, e.g., 3 to 5, base pairs upstream from these sequences. See, e.g., Horvath et al., SCIENCE 2010; 327(5962): 167-170, and Deveau et al, J BACTERIOL 2008; 190(4): 1390-1400. In some embodiments, an active Cas9 molecule of S. mutans recognizes the sequence motif NGG or NAAR (R-A or G) and directs cleavage of a core target nucleic acid sequence 1 to 10, e.g., 3 to 5 base pairs, upstream from this sequence. See, e.g., Deveau et al., J BACTERIOL 2008; 190(4): 1390-1400.

In some embodiments, an active Cas9 molecule of S. aureus recognizes the sequence motif NNGRR (R=A or G) and directs cleavage of a target nucleic acid sequence 1 to 10, e.g., 3 to 5, base pairs upstream from that sequence. See, e.g., Ran F. et al., NATURE, vol. 520, 2015, pp. 186-191. In some embodiments, an active Cas9 molecule of N. meningitidis recognizes the sequence motif NNNNGATT and directs cleavage of a target nucleic acid sequence 1 to 10, e.g., 3 to 5, base pairs upstream from that sequence. See, e.g., Hou et al., PNAS EARLY EDITION 2013, 1-6. The ability of a Cas9 molecule to recognize a PAM sequence can be determined, e.g., using a transformation assay described in Jinek et al, SCIENCE 2012, 337:816.

Exemplary naturally occurring Cas9 molecules are described in Chylinski et al, RNA Biology 2013; 10:5, 727-737. Such Cas9 molecules include Cas9 molecules of a cluster 1 bacterial family, cluster 2 bacterial family, cluster 3 bacterial family, cluster 4 bacterial family, cluster 5 bacterial family, cluster 6 bacterial family, a cluster 7 bacterial family, a cluster 8 bacterial family, a cluster 9 bacterial family, a cluster 10 bacterial family, a cluster 1 1 bacterial family, a cluster 12 bacterial family, a cluster 13 bacterial family, a cluster 14 bacterial family, a cluster 1 bacterial family, a cluster 16 bacterial family, a cluster 17 bacterial family, a cluster 1 8 bacterial family, a cluster 19 bacterial family, a cluster 20 bacterial family, a cluster 21 bacterial family, a cluster 22 bacterial family, a cluster 23 bacterial family, a cluster 24 bacterial family, a cluster 25 bacterial family, a cluster 26 bacterial family, a cluster 27 bacterial family, a cluster 28 bacterial family, a cluster 29 bacterial family, a cluster 30 bacterial family, a cluster 31 bacterial family, a cluster 32 bacterial family, a cluster 33 bacterial family, a cluster 34 bacterial family, a cluster 35 bacterial family, a cluster 36 bacterial family, a cluster 37 bacterial family, a cluster 38 bacterial family, a cluster 39 bacterial family, a cluster 40 bacterial family, a cluster 41 bacterial family, a cluster 42 bacterial family, a cluster 43 bacterial family, a cluster 44 bacterial family, a cluster 45 bacterial family, a cluster 46 bacterial family, a cluster 47 bacterial family, a cluster 48 bacterial family, a cluster 49 bacterial family, a cluster 50 bacterial family, a cluster 5 1 bacterial family, a cluster 52 bacterial family, a cluster 53 bacterial family, a cluster 54 bacterial family, a cluster 55 bacterial family, a cluster 56 bacterial family, a cluster 57 bacterial family, a cluster 58 bacterial family, a cluster 59 bacterial family, a cluster 60 bacterial family, a cluster 61 bacterial family, a cluster 62 bacterial family, a cluster 63 bacterial family, a cluster 64 bacterial family, a cluster 65 bacterial family, a cluster 66 bacterial family, a cluster 67 bacterial family, a cluster 68 bacterial family, a cluster 69 bacterial family, a cluster 70 bacterial family, a cluster 71 bacterial family, a cluster 72 bacterial family, a cluster 73 bacterial family, a cluster 74 bacterial family, a cluster 75 bacterial family, a cluster 76 bacterial family, a cluster 77 bacterial family, or a cluster 78 bacterial family.

Exemplary naturally occurring Cas9 molecules include a Cas9 molecule of a cluster 1 bacterial family. Examples include a Cas9 molecule of: S. pyogenes (e.g., strain SF370, MGAS 10270, MGAS 10750, MGAS2096, MGAS315, MGAS5005, MGAS6180, MGAS9429, NZ131 and SSI-1), S. thermophilus (e.g., strain LMD-9), S. pseudoporcinus (e.g., strain SPIN 20026), S. mutans (e.g., strain UA 159, NN2025), S. macacae (e.g., strain NCTC1 1558), S. gallolylicus (e.g., strain UCN34, ATCC BAA-2069), S. equines (e.g., strain ATCC 9812, MGCS 124), S. dysdalactiae (e.g., strain GGS 124), S. bovis (e.g., strain ATCC 700338), S. cmginosus (e.g.; strain F021 1), S. agalactia* (e.g., strain NEM316, A909), Listeria monocytogenes (e.g., strain F6854), Listeria innocua (L. innocua, e.g., strain Clip 11262), EtUerococcus italicus (e.g., strain DSM 15952), or Enterococcus faecium (e.g., strain 1, 23, 408). Additional exemplary Cas9 molecules are a Cas9 molecule of Neisseria meningitidis (Hou et al. PNAS Early Edition 2013, 1-6) and a S. aureus Cas9 molecule.

In some embodiments, a Cas9 molecule, e.g., an active Cas9 molecule or inactive Cas9 molecule, comprises an amino acid sequence: having 60%, 65%, 70%, 75%, 80%, 85%, 90%, 95%, 96%, 97%, 98%, or 99% homology with; differs at no more than 1%, 2%, 5%, 10%, 15%, 20%, 30%, or 40% of the amino acid residues when compared with; differs by at least 1, 2, 5, 10 or 20 amino acids but by no more than 100, 80, 70, 60, 50, 40 or 30 amino acids from; or is identical to; any Cas9 molecule sequence described herein or a naturally occurring Cas9 molecule sequence, e.g., a Cas9 molecule from a species listed herein or described in Chylinski et al., RNA Biology 2013, 10:5, I2I-T, 1 Hou et al. PNAS Early Edition 2013, 1-6.

In some embodiments, the Cas9 molecule is Cpf1, C2c1, C2c2, or C2c3 protein and modifications thereof. See, for example, Zetsche et al., Cell, 163: 1-13 (2015), the contents of which are herein incorporated by reference in their entirety, for a description of Cpf1, which is homologous to Cas9, and contains a RuvC-like nuclease domain. The Cpf1 sequences of Zetsche are incorporated by reference in their entirety. See, e.g., Zetsche, Tables 51 and S3; see also, e.g., Makarova et al., Nat Rev Microbiol, 13(11): 722-36 (2015); Shmakov et al., Molecular Cell, 60:385-397 (2015).

In some embodiments, a Cas9 molecule comprises an amino acid sequence having 60%, 65%, 70%, 75%, 80%, 85%, 90%, 95%, 96%, 97%, 98%, or 99% homology with; differs at no more than 1%, 2%, 5%, 10%, 15%, 20%, 30%, or 40% of the amino acid residues when compared with; differs by at least 1, 2, 5, 10 or 20 amino acids but by no more than 100, 80, 70, 60, 50, 40 or 30 amino acids from; or is identical to; S. pyogenes Cas9 (UniProt Q99ZW2). In some embodiments, the Cas9 molecule is a S. pyogenes Cas9 variant, such as a variant described in Slaymaker et al., Science Express, available online Dec. 1, 2015 at Science DOI: 10.1126/science.aad5227; Kleinstiver et al., Nature, 529, 2016, pp. 490-495, available online Jan. 6, 2016 at doi:10.1038/nature16526; or US2016/0102324, the contents of which are incorporated herein in their entirety. In some embodiments, the Cas9 molecule is catalytically inactive, e.g., dCas9. Tsai et al. (2014), Nat. Biotech. 32:569-577; U.S. Pat. Nos. 8,871,445; 8,865,406; 8,795,965; 8,771,945; and 8,697,359, the contents of which are hereby incorporated by reference in their entirety. A catalytically inactive Cas9, e.g., dCas9, molecule can be fused with a transcription modulator, e.g., a transcription repressor or transcription activator.

In some embodiments, the Cas9 molecule, e.g., a Cas9 of S. pyogenes, can additionally comprise one or more amino acid sequences that confer additional activity. In some aspects the Cas9 molecule can comprise one or more additional polypeptide domains, such as, for example, additional nuclease domains (e.g., FokI), or transcriptional activation or repression domains. Additional examples are described in, for example, Komor et al, Nature, 533(7603): 420-420; doi: 10.1038/nature17946, incorporated herein by reference in its entirety. In some aspects, the Cas9 molecule can comprise one or more nuclear localization sequences (NLSs), such as at least 1, 2, 3, 4, 5, 6, 7, 8, 9, 10, or more NLSs. Typically, an NLS consists of one or more short sequences of positively charged lysines or arginines exposed on the protein surface, but other types of NLS are known. Non-limiting examples of NLSs include an NLS sequence comprising or derived from: the NLS of the SV40 virus large T-antigen, having the amino acid sequence PKKKRKV (SEQ ID NO:1). Other suitable NLS sequences are known in the art (e.g., Sorokin, Biochemistry (Moscow) (2007) 72:13, 1439-1457; Lange J Biol Chem. (2007) 282:8, 5101-5). In any of the aforementioned embodiments, the Cas9 molecule can additionally (or alternatively) comprise a tag, e.g., a His tag, e.g., a His(6) tag or His(8) tag, e.g., at the N terminus or the C terminus.

Thus, engineered CRISPR gene editing systems, e.g., for gene editing in eukaryotic cells, typically involve (1) a guide RNA molecule (gRNA) comprising a targeting domain (which is capable of hybridizing to the genomic DNA target sequence), and sequence which is capable of binding to a Cas, e.g., Cas9 enzyme, and (2) a Cas, e.g., Cas9, protein. This second domain can comprise a domain referred to as a tracr domain. The targeting domain and the sequence which is capable of binding to a Cas, e.g., Cas9 enzyme, can be disposed on the same (sometimes referred to as a single gRNA, chimeric gRNA or sgRNA) or different molecules (sometimes referred to as a dual gRNA or dgRNA). If disposed on different molecules, each includes a hybridization domain which allows the molecules to associate, e.g., through hybridization.

gRNA molecule formats are known in the art. An exemplary gRNA molecule, e.g., dgRNA molecule, of the present invention comprises, e.g., consists of, a first nucleic acid having the sequence:

(SEQ ID NO: 2)
nnnnnnnnnnnnnnnnnnnnGUUUUAGAGCUAUGCUGUUUUG,

where the “n”'s refer to the residues of the targeting domain, e.g., as described herein, and can consist of 15-25 nucleotides, e.g., consists of 20 nucleotides;
and a second nucleic acid sequence having the exemplary sequence:
AACUUACCAAGGAACAGCAUAGCAAGUUAAAAUAAGGCUAGUCCGUUAUCAA CUUGAAAAAGUGGCACCGAGUCGGUGC, optionally with 1, 2, 3, 4, 5, 6, or 7 (e.g., 4 or 7, e.g., 7) additional U nucleotides at the 3′ end (SEQ ID NO:3).

The second nucleic acid molecule can alternatively consist of a fragment of the sequence above, wherein such fragment is capable of hybridizing to the first nucleic acid. An example of such second nucleic acid molecule is:

AACAGCAUAGCAAGUUAAAAUAAGGCUAGUCCGUUAUCAACUUGAAAAAGUG GCACCGAGUCGGUGC, optionally with 1, 2, 3, 4, 5, 6, or 7 (e.g., 4 or 7, e.g., 7) additional U nucleotides at the 3′ end (SEQ ID NO:4).

Another exemplary gRNA molecule, e.g., a sgRNA molecule, of the present invention comprises, e.g., consists of a first nucleic acid having the sequence: nnnnnnnnnnnnnnnnnnnGUUUUAGAGCUAGAAAUAGCAAGUUAAAAUAAGGCUAGU CCGUUAUCAACUUGAAAAAGUGGCACCGAGUCGGUGC (SEQ ID NO:5), where the “n”'s refer to the residues of the targeting domain, e.g., as described herein, and can consist of 15-25 nucleotides, e.g., consist of 20 nucleotides, optionally with 1, 2, 3, 4, 5, 6, or 7 (e.g., 4 or 7, e.g., 4) additional U nucleotides at the 3′ end.

Additional components and/or elements of CRISPR gene editing systems known in the art, e.g., are described in U.S. Publication No. 2014/0068797, WO2015/048577, and Cong (2013) Science 339: 819-823, the contents of which are hereby incorporated by reference in their entirety. Such systems can be generated which inhibit a target gene, by, for example, engineering a CRISPR gene editing system to include a gRNA molecule comprising a targeting domain that hybridizes to a sequence of the target gene. In some embodiments, the gRNA comprises a targeting domain which is fully complementarity to 15-25 nucleotides, e.g., 20 nucleotides, of a target gene. In some embodiments, the 15-25 nucleotides, e.g., 20 nucleotides, of the target gene, are disposed immediately 5′ to a protospacer adjacent motif (PAM) sequence recognized by the RNA-guided nuclease, e.g., Cas protein, of the CRISPR gene editing system (e.g., where the system comprises a S. pyogenes Cas9 protein, the PAM sequence comprises NGG, where N can be any of A, T, G or C).

In some embodiments, the gRNA molecule and RNA-guided nuclease, e.g., Cas protein, of the CRISPR gene editing system can be complexed to form a RNP complex. Such RNP complexes can be used in the methods and apparatus described herein. In other embodiments, nucleic acid encoding one or more components of the CRISPR gene editing system can be used in the methods and apparatus described herein.

In some embodiments, foreign DNA can be introduced into the cell along with the CRISPR gene editing system, e.g., DNA encoding a desired transgene, with or without a promoter active in the target cell type. Depending on the sequences of the foreign DNA and target sequence of the genome, this process can be used to integrate the foreign DNA into the genome, at or near the site targeted by the CRISPR gene editing system. For example, 3′ and 5′ sequences flanking the transgene can be included in the foreign DNA which are homologous to the gene sequence 3′ and 5′ (respectively) of the site in the genome cut by the gene editing system. Such foreign DNA molecule can be referred to “template DNA.”

In some embodiments, the CRISPR gene editing system of the present invention comprises Cas9, e.g., S. pyogenes Cas9, and a gRNA comprising a targeting domain which hybridizes to a sequence of a gene of interest. In some embodiments, the gRNA and Cas9 are complexed to form a RNP. In some embodiments, the CRISPR gene editing system comprises nucleic acid encoding a gRNA and nucleic acid encoding a Cas protein, e.g., Cas9, e.g., S. pyogenes Cas9. In some embodiments, the CRISPR gene editing system comprises a gRNA and nucleic acid encoding a Cas protein, e.g., Cas9, e.g., S. pyogenes Cas9.

Additional CRISPR gene editing systems which can be used with the present methods and apparatus include CRISPR gene editing systems described in, for example, international application publication WO2017/115268 and, for example, international application publication WO2017/093969, the contents of each of which are hereby incorporated by reference in its entirety.

TALEN Gene Editing Systems

TALENs are produced artificially by fusing a TAL effector DNA binding domain to a DNA cleavage domain. Transcription activator-like effects (TALEs) can be engineered to bind any desired DNA sequence, e.g., a target gene. By combining an engineered TALE with a DNA cleavage domain, a restriction enzyme can be produced which is specific to any desired DNA sequence, including a HLA or TCR sequence. These can then be introduced into a cell, wherein they can be used for genome editing. Boch (2011) Nature Biotech. 29: 135-6; and Boch et al. (2009) Science 326: 1509-12; Moscou et al. (2009) Science 326: 3501.

TALEs are proteins secreted by Xanthomonas bacteria. The DNA binding domain contains a repeated, highly conserved 33-34 amino acid sequence, with the exception of the 12th and 13th amino acids. These two positions are highly variable, showing a strong correlation with specific nucleotide recognition. They can thus be engineered to bind to a desired DNA sequence.

To produce a TALEN, a TALE protein is fused to a nuclease (N), which is, for example, a wild-type or mutated FokI endonuclease. Several mutations to FokI have been made for its use in TALENs; these, for example, improve cleavage specificity or activity. Cermak et al. (2011) Nucl. Acids Res. 39: e82; Miller et al. (2011) Nature Biotech. 29: 143-8; Hockemeyer et al. (2011) Nature Biotech. 29: 731-734; Wood et al. (2011) Science 333: 307; Doyon et al. (2010) Nature Methods 8: 74-79; Szczepek et al. (2007) Nature Biotech. 25: 786-793; and Guo et al. (2010)J Mol. Biol. 200: 96.

The FokI domain functions as a dimer, requiring two constructs with unique DNA binding domains for sites in the target genome with proper orientation and spacing. Both the number of amino acid residues between the TALE DNA binding domain and the FokI cleavage domain and the number of bases between the two individual TALEN binding sites appear to be important parameters for achieving high levels of activity. Miller et al. (2011) Nature Biotech. 29: 143-8.

A TALEN (or pair of TALENs) can be used inside a cell to produce a double-stranded break (DSB). A mutation can be introduced at the break site if the repair mechanisms improperly repair the break via non-homologous end joining. For example, improper repair can introduce a frame shift mutation. Alternatively, foreign DNA can be introduced into the cell along with the TALEN, e.g., DNA encoding a transgene, and depending on the sequences of the foreign DNA and chromosomal sequence, this process can be used to integrate the transgene at or near the site targeted by the TALEN. TALENs specific to a target gene can be constructed using any method known in the art, including various schemes using modular components. Zhang et al. (2011) Nature Biotech. 29: 149-53; Geibler et al. (2011) PLoS ONE 6: e19509; U.S. Pat. Nos. 8,420,782; 8,470,973, the contents of which are hereby incorporated by reference in their entirety.

Zinc Finger Nuclease (ZFN) Gene Editing Systems

“ZFN” or “Zinc Finger Nuclease” refer to a zinc finger nuclease, an artificial nuclease which can be used to modify, e.g., delete one or more nucleic acids of, a desired nucleic acid sequence.

Like a TALEN, a ZFN comprises a FokI nuclease domain (or derivative thereof) fused to a DNA-binding domain. In the case of a ZFN, the DNA-binding domain comprises one or more zinc fingers. Carroll et al. (2011) Genetics Society of America 188: 773-782; and Kim et al. (1996) Proc. Natl. Acad. Sci. USA 93: 1156-1160.

A zinc finger is a small protein structural motif stabilized by one or more zinc ions. A zinc finger can comprise, for example, Cys2His2, and can recognize an approximately 3-bp sequence. Various zinc fingers of known specificity can be combined to produce multi-finger polypeptides which recognize about 6, 9, 12, 15 or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells.

Like a TALEN, a ZFN must dimerize to cleave DNA. Thus, a pair of ZFNs are required to target non-palindromic DNA sites. The two individual ZFNs must bind opposite strands of the DNA with their nucleases properly spaced apart. Bitinaite et al. (1998) Proc. Natl. Acad. Sci. USA 95: 10570-5.

Also like a TALEN, a ZFN can create a double-stranded break in the DNA, which can create a frame-shift mutation if improperly repaired, leading to a decrease in the expression and amount of the target gene in a cell. ZFNs can also be used with homologous recombination to mutate the target gene or locus, or to introduce nucleic acid encoding a desired transgene at a site at or near the targeted sequence.

ZFNs specific to sequences in a target gene can be constructed using any method known in the art. See, e.g., Provasi (2011) Nature Med. 18: 807-815; Torikai (2013) Blood 122: 1341-1349; Cathomen et al. (2008) Mol. Ther. 16: 1200-7; and Guo et al. (2010) J. Mol. Biol. 400: 96; U.S. Patent Publication 2011/0158957; and U.S. Patent Publication 2012/0060230, the contents of which are hereby incorporated by reference in their entirety. In some embodiments, The ZFN gene editing system can also comprise nucleic acid encoding one or more components of the ZFN gene editing system.

The above gene editing methods can be applied to a variety of different target cells. For example, the target cell can be a mammalian cell, e.g., a human cell. The cell can be of a desired type, such as a progenitor cell, an immune effector cell (e.g., a T cell or NK cell), a human embryonic stem cell (hES cell), an induced pluripotent stem cell (iPS), a mesenchymal stem cell, a keratinocyte or a human bronchial epithelial cell, among others. In some embodiments, the cell is a human T cell. In some embodiments, the cell is a human hematopoietic stem and progenitor cell (HSPC). In some embodiments the cell is autologous to the subject to which it can be administered. In other embodiments, the cell is allogeneic to the subject to which it can be administered.

FIG. 3B schematically depicts an example of various steps of a cycle in an exemplary clinical use of gene editing techniques according to the present teachings for performing gene therapy. FIG. 3C schematically illustrates another example of various procedures of a cycle in an exemplary clinical use of gene editing techniques according to the present teachings for performing gene therapy. In the examples shown in FIGS. 3B-3C, a sample of cells (e.g., bone marrow (as shown in FIG. 3C) or peripheral blood) of a patient (donor patient) (e.g., a patient suffering from a genetic disease) is collected and the hematopoietic stem and progenitor cells (HSPC), e.g., CD34+ cells, are isolated, e.g., using antibody and/or size selection. The methods according the present teachings are employed to deliver a gene editing complex (e.g., a Cas9-gRNA complex) to the HSC cells for effecting a genetic correction. The modified cells are then collected, e.g., for quality control, and optionally cyropreserved. The collected cells are then prepared for transplantation into a patient (recipient patient). In some embodiments, the patient is the same as the patient from which the cells are collected, e.g., the transplantation is autologous. In other embodiments, the cells can be collected from a healthy donor patient and delivered to a different recipient patient, e.g., a patient suffering from a genetic disease, e.g., the transplantation is allogeneic. Similar methods can be utilized for the clinical use of other cell types, such as T cells, by collecting a population of cells comprising the desired cell population from a donor patient.

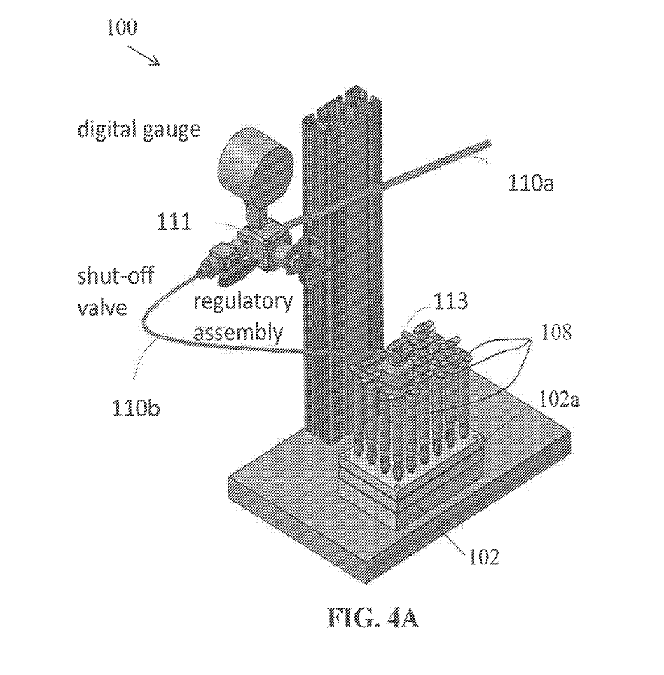

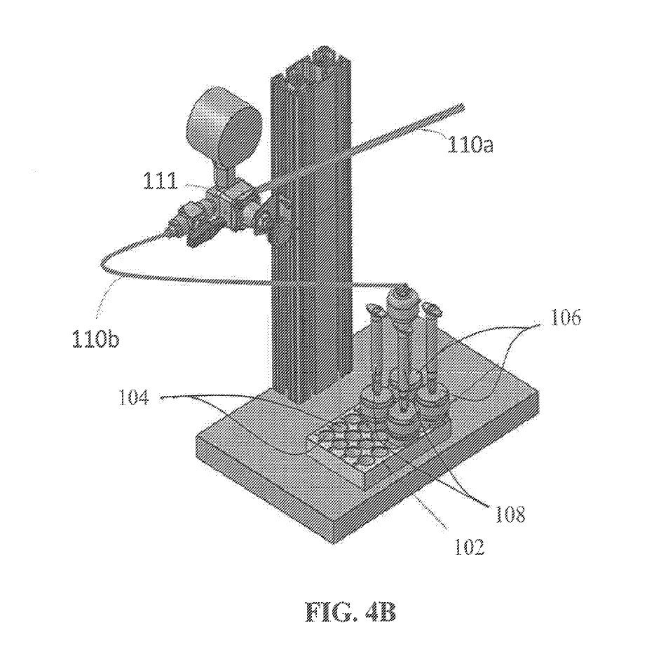

The above methods for delivering agents into cells can be implemented using a variety of different systems. For example, FIGS. 4A and 4B schematically depict a system 100 according to an embodiment of the present teachings for cell processing, which includes a collection block 102 (herein also referred to as a collection plate) that includes a plurality of wells 104 for receiving processed cells. While in this embodiment the collection plate includes 24 wells, in other embodiments the number of the wells provided in the collection plate can be different.

The system 100 further includes a plurality of cell processing assemblies 106, each of which is fluidly coupled to one of the wells 104 in the collection block 102 (in FIG. 4A the cell processing assemblies 106 are hidden by cover 102a; FIG. 4B depicts the cell processing assemblies 106 with the cover 102a removed). As discussed in more detail below, each of the cell processing assemblies can receive a fluid carrier (typically a liquid carrier) in which a plurality of cells and one or more agents, such as those discussed above, are entrained. The passage of a cell through a cell processing assembly 106 can result in a modulation of the cell's physical and/or chemical attributes, e.g., a change in the permeability of the cell's membrane, which can in turn allow the uptake of the agent(s) by the cell.

In some embodiments, the system can include a plurality of syringes 108, where each syringe can be coupled to one of the cell processing assemblies 106 for delivering a sample containing a mixture (e.g., a solution) of the fluid carrier, a plurality of target cells and one or more agent(s) to a corresponding cell processing assembly. More specifically, the sample can be disposed in a syringe, and a positive pressure interface can be used to apply a pressure to the sample to push it through a cell processing assembly coupled to the syringe.

More specifically, in this embodiment, a gas line 110a transfers a gas (e.g., nitrogen) from a source (not shown) to a regulator assembly 111, which regulates the gas pressure for application to a positive pressure interface 113 via a gas line 110b. The positive pressure interface 113 creates a seal on the syringe barrel and provides a regulated gas pressure to a sample in the syringe. In some embodiments, a pressure of at least about 5 psi, e.g., in a range of about 5 psi to about 100 psi, can be applied to the sample in the syringe to cause its passage through a cell processing assembly, e.g., at a flow rate greater than about 20 mL/min. In general, the pressure applied to a sample, e.g., via an applied gas pressure, can be adjusted so as to vary the flow rate of the sample through the cell processing assembly. In some embodiments, the system 100 allows processing the cells at a rate of at least about 4 billion cells/min.

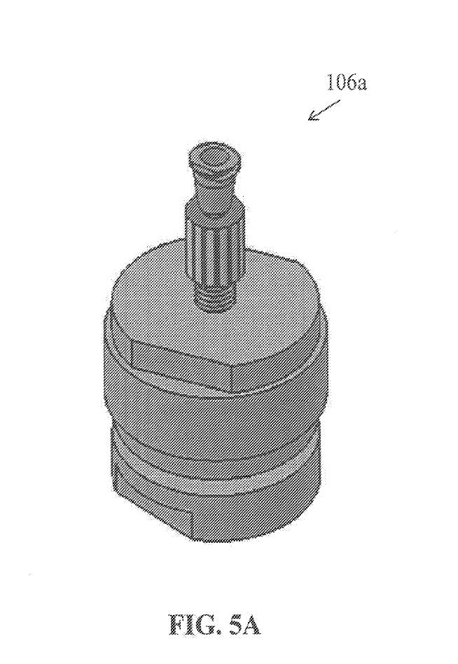

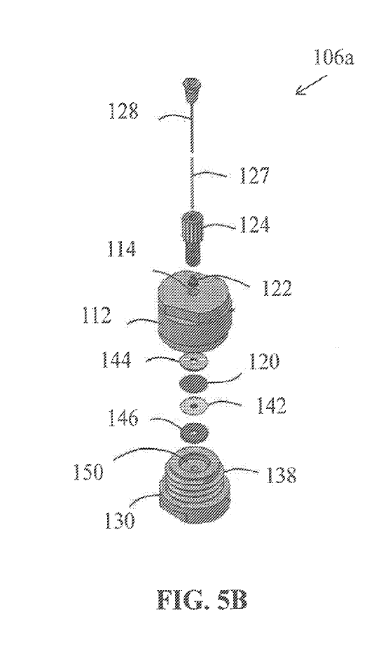

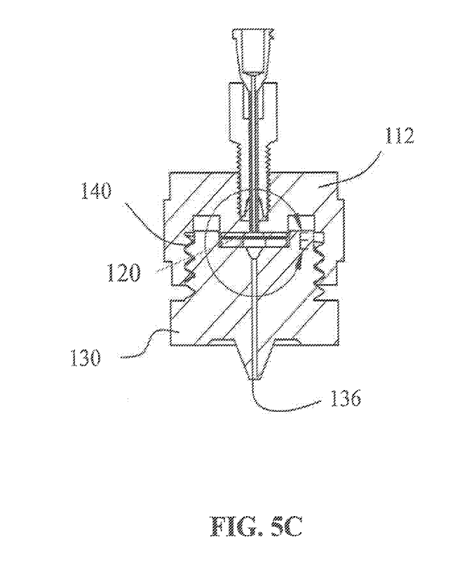

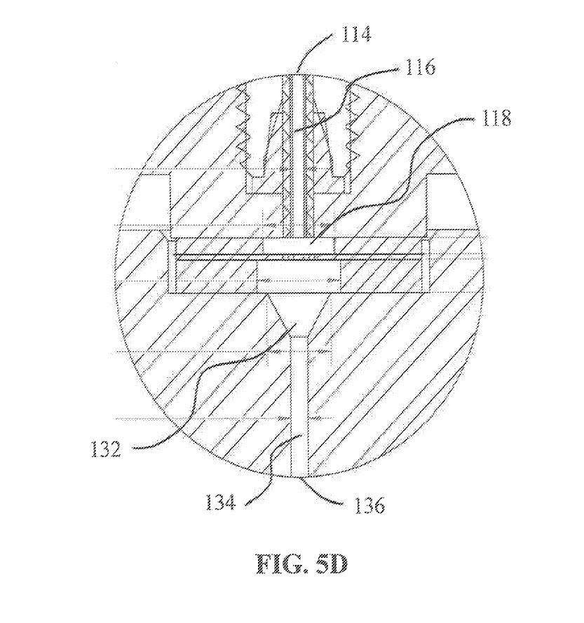

With reference to FIGS. 5A, 5B, 5C, and 5D, each of cell processing assemblies 106 (e.g., the illustrative cell processing assembly 106a) can include an input block 112 having an inlet port 114 that allows delivery of a sample to the inlet block 112. The inlet block 112 further includes a fluid passage 116 that extends from the inlet port 114 to an input chamber 118 below which a porous membrane 120 is positioned, as discussed in more detail below. The volume of the input chamber 118 can be, for example, in a range of about 50 μL to about 1 L, though other volumes can also be used.

In this embodiment, a ferrule 122 allows fluidly coupling the inlet port 114 of the illustrative cell processing assembly 106a, via a fitting 124 (e.g., a polyetheretherketone (PEEK) fitting) and a tubing 126, to a needle 128 of a syringe containing the sample. More specifically, the inlet port can include internal threads that can engage with the external threads of the fitting. The needle can extend through central openings of the fitting and the ferrule to fluidly couple the syringe to the inlet port 114.

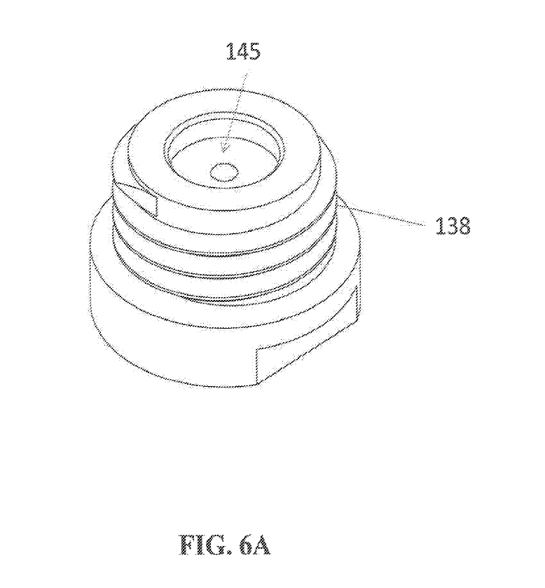

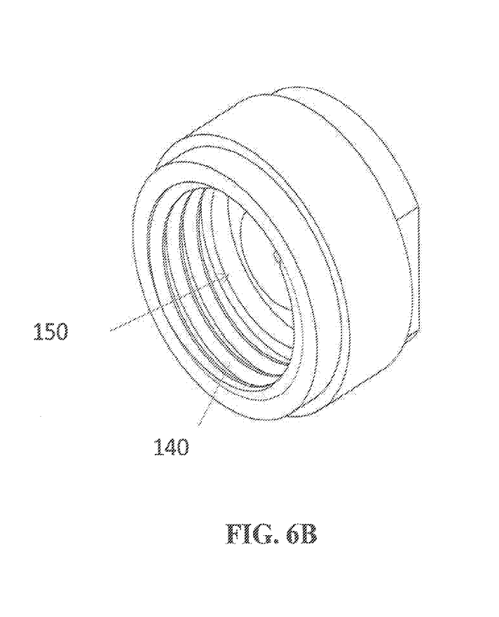

The cell processing assembly 106a further includes an output block 130 having an output chamber 132 that is fluidly coupled to a proximal end of a passageway 134 providing an outlet port 136 at a distal end thereof. In some embodiments, the output chamber 132 is configured to reduce dead volume and to facilitate the transfer of a sample to a respective collection well. For example, the output chamber 132 can have a suitable volume selected, for example, based on the inner diameters of the membrane 120, a mesh 142 and a washer 146 discussed below. For example, the volume of the output chamber 132 can be about 7.99 cubic millimeters, though other sizes can also be used. The fluid passageway 134 can have any suitable length and volume, e.g., a length of about 21.32 mm and a hole diameter of about 0.9 mm corresponding to a volume of about 13.56 cubic millimeters. The output block 132 includes a plurality of external threads 138 (see also FIG. 6A) that can engage with a plurality of internal threads 140 of the input block (see also FIG. 6B) to connect the two blocks together.

As noted above, the cell processing assembly 106a further includes a porous membrane 120 that is disposed between the input block and the output block so as to receive a sample containing cells and one or more agents to be internalized by the cells via the input block and to allow at least a portion of the cells passing therethrough to reach the output block. A mesh 142, e.g., a stainless mesh in this embodiment, is disposed adjacent the porous membrane 120 for providing mechanical support thereto. In particular, the mesh can help the thin membrane withstand the passage of a liquid sample under pressure therethrough. A washer 144 received by a recess 145 (see FIG. 6A) of the input block and another washer 146 received by a recess 150 of the output block (see also FIG. 6B) facilitate fixedly positioning the porous membrane 120 and the mesh 142 between the input and the output blocks.

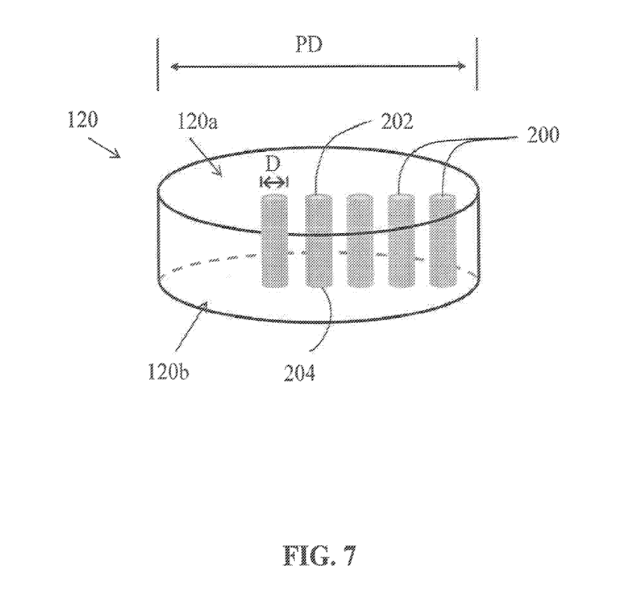

With reference to FIG. 7, in this embodiment, the porous membrane 120 has an essentially planar structure and a circular cross-section with a diameter (PD) in a range of about 1 mm to about 142 mm, though other diameters can also be employed. The porous membrane 120 includes a plurality of pores 200 that extend from a top surface 120a to a bottom surface 120b of the membrane. In other words, each port extends from an inlet port (e.g., inlet port 202) that is substantially flush with the top surface of the membrane 120 to an outlet port (e.g., outlet port 204) that is substantially flush with the bottom surface of the membrane 120. Hence, in this embodiment, the thickness of the membrane is substantially identical to the length of the pores, which can be in a range of about 5 microns to about 30 microns. While in this embodiment, the pores have a substantially uniform length, in other embodiments, the lengths of the pores can be non-uniform. In such embodiments, the thickness of the membrane can be non-uniform to conform to the lengths of the pores.

In this embodiment, each of the pores 200 is substantially cylindrical with a circular cross-section that is substantially uniform along the length of the pore. In general, the cross-sectional diameter D of each pore (which corresponds to the maximum cross-sectional dimension of the pore in this embodiment) is selected to be less than the diameter of a cell that is intended to undergo a change via passage through the pores so as to uptake one or more agents in the fluid carrier. In this embodiment, the cross-sectional diameter D of each pore can be less than about 40 microns, or less than about 30 microns, or less than about 20 microns, or less than about 15 microns, or less than about 10 microns. By way of example, the cross-sectional diameter D can be in a range of about 2 microns to about 40 microns, or in a range of about 2 microns to about 30 microns, or in a range of about 2 microns to about 20 microns, or in a range of about 2 microns to about 15 microns, or in a range of about 2 microns to about 10 microns. In some embodiments, the cross-sectional diameter D can be in a range of about 5 microns to about 8 microns.

The thickness of the membrane (which in this embodiment corresponds to the length of the pores) can be, for example, in a range of about 2 microns to about 40 microns, or in a range of about 2 microns to about 20 microns, or in a range of about 2 microns to about 30 microns, or in a range of about 2 microns to about 20 microns, or in a range of about 2 microns to about 10 microns. In some embodiments, the surface density of the pores can be, for example, in a range of about 1×10⁵ to about 2×10⁶ pores/cm². The surface density of the pores can be determined by dividing the number of pores by the area of the top or the bottom surface of the porous membrane. The high pore density advantageously allows efficient delivery and processing of the cells via their passage, under an applied pressure, through the porous membrane. For example, in some embodiments, the porous membrane can be used to transfect cells with an agent at a rate of at least about 4 billion cells per minute.

The porous membrane can be formed of a variety of materials, such as a polymer, a semiconductor or a metal. In many embodiments, the porous membrane is formed of a polymeric material. Some examples of suitable polymers include, without limitation, polycarbonate, polytetrafluoroethylene (PTFE), polystyrene, polyvinylidene fluoride (PVDF), polyethylene terephthalate (PET), poly methyl methacrylate (PMMA), polypropylene (PP), polyimide (PI), cyclic olefin copolymer (COC), cyclo olefin polymer (COP), polyester and polydimethylsiloxane (PDMS).

The pores of the porous membrane 120 can be generated using a variety of different methods. By way of example, the pores can be generated using any of ion track etching, laser drilling, plasma etching, or photolithography.

In some embodiments, at least a portion of the exposed surfaces of the membrane, and in many embodiments the entire exposed surface of a membrane, can be coated with a material. For example, the exposed surfaces of the membrane, including the inner surfaces of the pores, can be coated with a polymeric material, such as polyvinylpyrrolidone, polyethylene glycol (PEG) and PEG-based compounds, polyvinyl alcohol (PVA), hydroxyethylcellulose, tween 20, octadecylsilane, Brij-35, poly(p-xylenes), fluoropolymers, Pluronic F127. While in some cases the inner surfaces of the pores can be hydrophilic in other embodiments the inner surfaces of the pores can be hydrophobic. In some embodiments, a material coating the surfaces of the membrane can be selected so as to enhance the hydrophilicity or hydrophobicity of those surfaces.

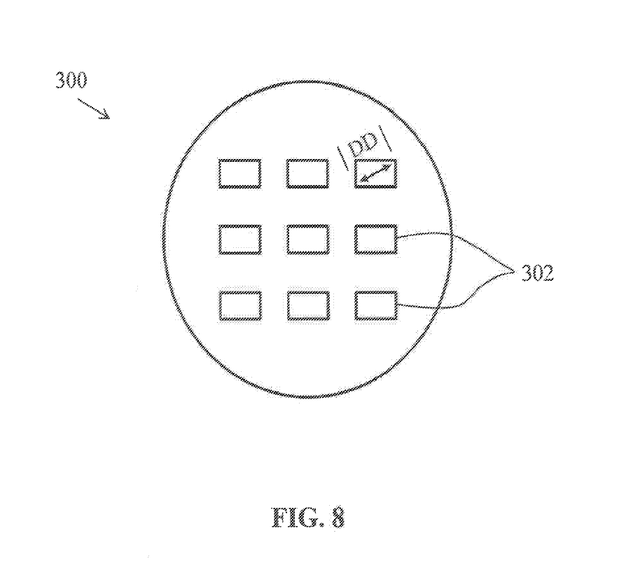

In other embodiments, the pores can have non-circular cross-sectional shapes, including regular and irregular shapes. By way of example, FIG. 8 schematically depicts a top view of another porous membrane 300 having a plurality of pores 302 with rectangular cross-sections. In this case, the dimension of the diagonal of the rectangle (DD) corresponds to the maximum cross-sectional dimension of a pore and is less than a threshold suitable for effecting a desired change in a cell as the cell passes through one of the pores. For example, the diagonal DD can be less than about 40 microns, or less than about 30 microns, or less than about 20 microns, or less than about 15 microns, or less than about 10 microns. Similar to the previous embodiment, the length of the holes can be, e.g., in a range of about 5 microns to about 30 microns, e.g., in a range of about 7 microns to about 21 microns.

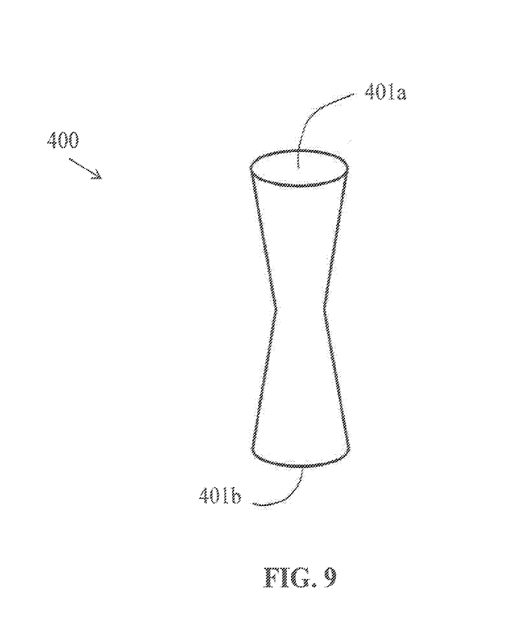

In some embodiments, the cross-sectional area of one or more of the pores can not be uniform along the entire length of the pore. By way of example, FIG. 9 schematically depicts such a pore 400 that extends from a proximal opening 401a to a distal opening 401b. The cross-sectional area of the pore decreases progressively from the proximal opening and reaches a minimum value at the middle of the pore and then increases from the middle of the pore to its distal end. Other variations of the cross-sectional area of the pore can also be employed in other embodiments.

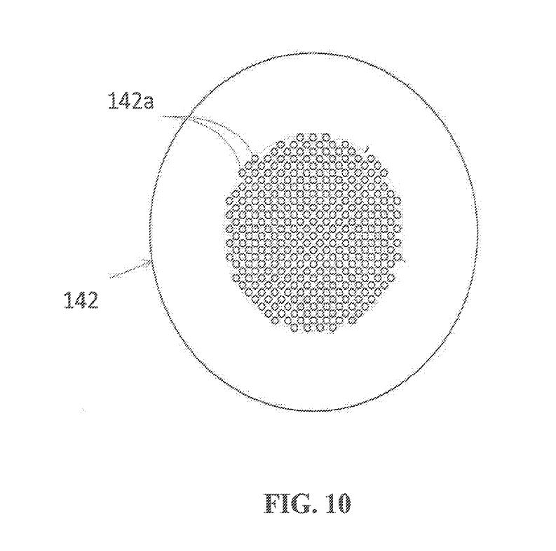

Referring to FIG. 5B as well as FIG. 10, the support mesh 142 is placed adjacent the membrane 120 to provide mechanical stability to the membrane, e.g., to help the membrane withstand the pressures applied thereto due to the passage of a liquid sample therethrough. In this embodiment, the mesh 142 is formed of stainless steel, though in other embodiments it can be formed of other materials. The mesh 142 includes a plurality of openings 142a, which have significantly larger cross-sectional diameters than those of the pores of the porous membrane 120. By way of example, the diameters of the mesh openings can be in a range of about 1 mm to about 5 mm. The cells exiting the porous membrane pass through the holes of the mesh to reach the output block 130.

As noted above, in many embodiments, the porous membrane is formed of a polymeric material and the mesh can be formed of suitable metal, e.g., stainless steel. The other components of the system, e.g., the input and output block, can be formed of any suitable material, e.g., a polymeric material.

Referring again to FIGS. 4A, 4B, 5A and 5B, in use, a plurality of samples, each containing a fluid carrier (e.g., a buffer solution), a plurality of cells of interest and one or more agents to be internalized by the cells, can be introduced via one or more of the syringes 108 into one or more of the cell processing assemblies 106. The pressure applied to the samples by the pistons 110 can facilitate their passage through the pores of the porous membrane 120. As a cell passes through one of the pores 200, the wall of the pore applies a compressive pressure to the cell because the cross-sectional dimension of the pore is less than the diameter of the cell. Without being limited to any particular theory, such a compressive pressure can cause a change in the cell, e.g., it can change the permeability of the cell's membrane. And such a cellular change can in turn facilitate the uptake of the agent(s) by the cell. In many embodiments, the systems and the methods of the invention allow cellular uptake of many agent(s) that cannot be normally internalized by a cell, e.g., because the cell's membrane is not permeable to those agents under normal condition.

After passage through one of the porous membrane 120, the sample, including the cells entrained in the fluid carriers, pass through the openings of one of the meshes 142 to reach the output block 130. The sample can then pass through the outlet of the outlet 136 of the output block 130 to be collected in one of the wells 104 of the collection block 102.

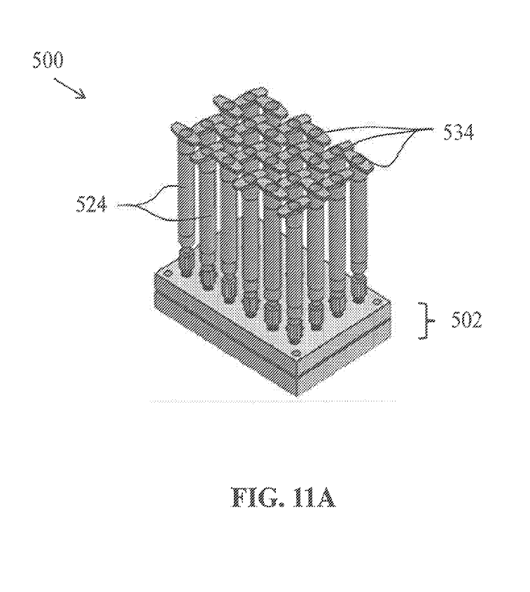

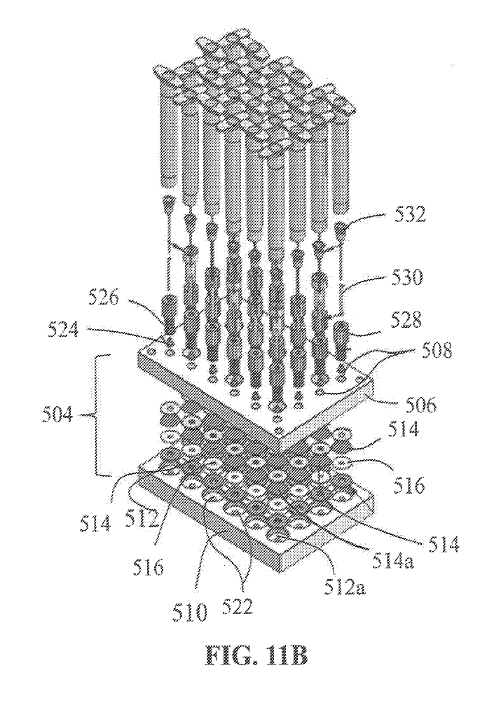

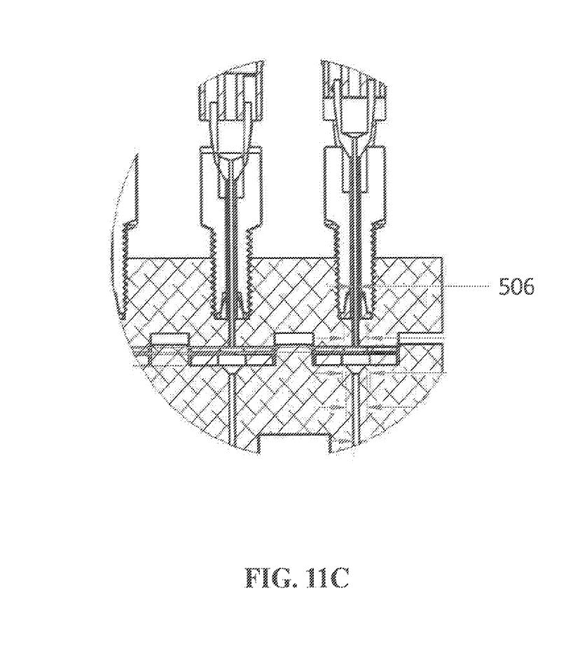

FIGS. 11A, 11B, and 11C schematically depict a system 500 according to another embodiment of the present teachings for cell processing. The system 500 includes a cell processing assembly 502 that includes a plurality of parallel channels 504 for concurrently processing a plurality of cells. The cell processing assembly 502 includes an inlet support element (e.g., a plate) 506 having a plurality of inlet ports 508 through which a plurality of samples, each containing cells and one or more agents to be delivered to the cells, can be introduced into the assembly. The cells are typically entrained in a liquid carrier (e.g., a buffer solution) together with one or more agents to be internalized by the cells. While in this embodiment, the input plate includes 24 inlet ports (as shown more clearly in FIG. 11A), in other embodiments the number of inlet ports can be different, e.g., based on the demands of a particular application.

The illustrated cell processing assembly 502 further includes an outlet support element (e.g., a plate) 510 (herein also referred to as the outlet block) that includes a plurality of wells 512 for receiving the processed samples. As discussed in more detail below, each sample-receiving well 512 is in fluid communication with one of the inlet ports.

The inlet and the outlet blocks can be formed of a variety of different materials. Some examples of suitable materials include, without limitation, 316 stainless steel, polyether ether ketone (PEEK) polymer, among others. Further, the inlet and outlet plates can have a variety of different thicknesses, e.g., a thickness in a range of about 10 to about 20 mm.

A plurality of porous membranes 514 is disposed between the inlet plate 506 and the outlet plate 510 such that each porous membrane is positioned relative to an inlet port of the inlet block to receive cells introduced into the assembly via that inlet port and is positioned relative to a respective well of the outlet block such that the cells passing through the membrane are received in that well. By way of example, the porous membrane 514a is positioned relative to the inlet port 508a of the inlet block so as to receive a sample containing cells that is introduced into the assembly via the inlet port 508a and the porous membrane 514a is positioned relative to the well 512a such that the sample would reach that well after its passage through the membrane.

The porous membranes can have the properties discussed above in connection with the previous embodiments. For example, each of the porous membranes can have a maximum cross-sectional dimension that is less than the diameters of cells for which the system 500 is intended to provide cell processing (e.g., cell transfection). By way of example, the maximum cross-sectional dimension of the pores of the porous membranes 514 can be in a range of about 2 microns to about 40 microns, or in a range of about 2 microns to about 30 microns, or in a range of about 2 microns to about 20 microns, or in a range of about 2 microns to about 15 microns, or in a range of about 2 microns to about 10 microns, e.g., in a range of about 5 microns to about 8 microns. Similar to the previous embodiments, in this embodiment, the inlet of each pore of a porous membrane is substantially flush with an input surface of the membrane and the outlet of each pore is substantially flush with an output surface of the membrane. Further, the porous membranes 514 can be formed of a variety of different polymeric materials, such as those discussed above.

With reference to FIG. 11B, the system 500 further includes a plurality of meshes 516 (e.g., stainless meshes), each of which is disposed adjacent one of the porous membranes 514. As discussed above in connection with the previous embodiments, the meshes can provide mechanical support to the porous membranes. The meshes can have pores in a range of about 1 mm to about 5 mm, which allow facile passage of the cells that pass through the pores of the membranes 514. Further, as discussed above, in some embodiments, the meshes 516 can have a thickness, e.g., in a range of about 0.15 mm to about 0.25 mm, though other thicknesses can also be employed.

An inlet seal 518 and an outlet seal 520, e.g., a Teflon® washers, are employed to provide a fluid-tight seal between the inlet block 506 and the outlet block 510. The outlet seals are received in respective recesses 522 provided in the outlet plate and the inlet seals are received in respective recesses (not shown) provided in the inlet plate 506 (see FIG. 11C).

Similar to the previous embodiment, in this embodiment, a plurality of syringes 524 are provided, each of which is fluidly coupled to one of the inlet ports of the inlet plate 506 to introduce a sample containing cells and one or more agents (the cells and the agent(s) are typically entrained in a liquid carrier) into the cell processing assembly. More specifically, in this embodiment, each syringe is coupled to a respective inlet port via one of the ferrules 524, one of the threaded fittings 526, one of the socket head cap screws (SHCS) 528, one of the tubings 530, and one of the fittings 532. A plurality of pistons 534, each of which is coupled to one of the syringes 524 allow pushing the samples contained in the syringes onto the porous membranes 514. In other embodiments, pressurized gas can be employed to push the samples contained in the syringes onto the porous membrane.

Accordingly, the cell processing system 500 includes multiple parallel cell processing channels (24 channels in this embodiment), each of which extends from one of the inlet ports via a porous membrane to one of the outlet ports, for concurrent processing of a plurality of cells. By way of example, in some cases, each unit of the cell processing system 500 can be employed to process at least about 4 billion cells/min.

More specifically, in use, a plurality of samples, each of which contains cells and one or more agents to be internalized by the cells, which are typically entrained in a liquid carrier, can be concurrently introduced into the cell processing assembly, e.g., via application of pressure to the pistons of the syringes. In this embodiment, the cell processing assembly 502 includes 24 parallel cell processing channels, though in other embodiments other numbers of parallel cell processing channels can be provided. Similar to the previous embodiment, in some cases, the pressure applied to the samples contained in the syringes can be greater than about 5 psi. In some cases, the flow rate of the sample through each cell processing channel can be at least about 20 mL/min. The total flow rate of the samples through the cell processing assembly can be obtained by multiplying the flow rate through each cell processing channel by the number of those channels.

The passage of the cells through each of the porous membranes can cause a change in at least some of those cells such that the cells can uptake one or more agents present in the fluid carrier By way of example and without being limited to any particular theory, as discussed above, the passage of a cell through one of the pores of one of the membranes can subject the cell to compressive forces due to small cross-sectional dimension of the pore, which can in turn cause a change (e.g., a transient change in the permeability of the cell's membrane) that allows the uptake of one or more of the agents present in the carrier by the cell.

After passage through the porous membranes and the respective meshes, the cells, including those that have undergone a transformation via passage through the membranes, pass through the outlet ports provided in the outlet plate to reach the collection wells provided in the output plate 510.

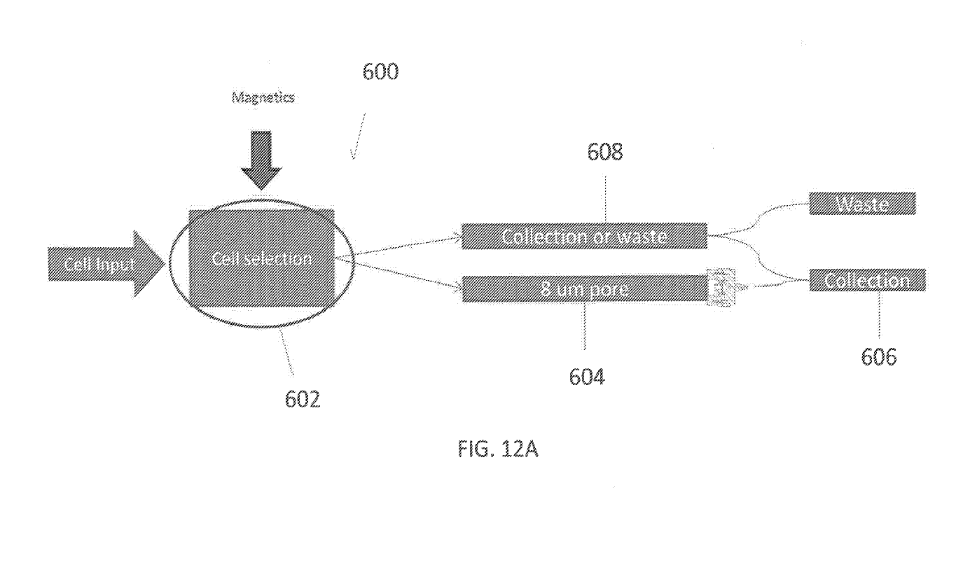

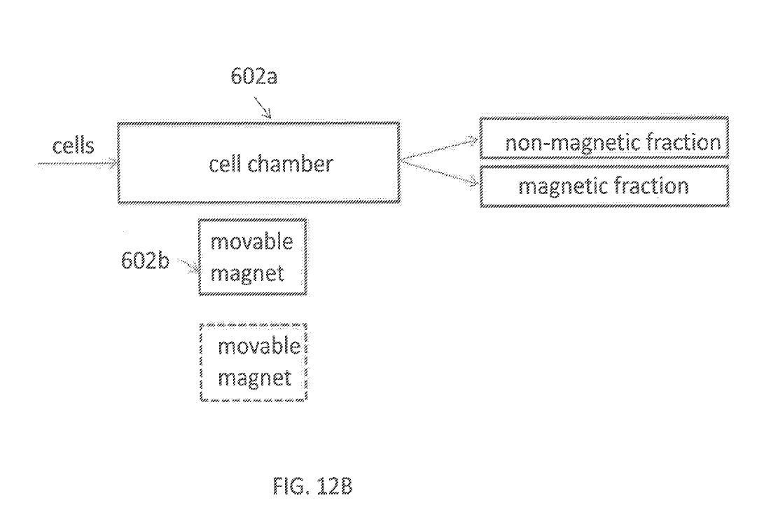

As noted above, in some embodiments, one or more cells having desired characteristics are selected from a collection of heterogeneous cells and the selected cells are then processed in a manner described above to transfect them with one or more agents. By way of example, FIG. 12A schematically depicts a system 600 according to an embodiment of the invention, which includes a cell selection (sorting) device 602 that can receive a collection of heterogeneous cells. The device 602 can sort the received cells based on one or more desired characteristics. For example, the device 602 can sort the cells based on the preferential presence of a surface protein marker (e.g., a marker such as CD34, CD90, CD49f and combinations thereof), cell size, or other cellular characteristics. The system 600 further includes a cell processing device 602 according to the present teachings for transfecting cells with one or more agents of interest, which is coupled to the cell sorting device 602 to receive the selected cells therefrom. In this embodiment, the cell processing device 602 includes a porous membrane (not shown) with a pore size of about 8 microns through which the cells pass in presence of one or more agents. As discussed above, the passage of the cells through the porous membrane can mediate the uptake of the agent(s) by the cells. A collection unit 606 can collect the processed cells. The rejected cells (i.e., the cells that are not selected for further processing) can be collected by a waste collection unit 608 be discarded as waste.

The cell sorting device 602 can be implemented in a variety of different ways. For example, as shown schematically in FIG. 12B, the device 602 can include a cell chamber 602a and a magnet 602b movable relative to the cell chamber for separating cells bound to magnetic particles from unbound cells. The magnetic forces applied to the cells bound to magnetic particles can be modulated by moving the magnet 602a closer or farther from the cell chamber. More specifically, upon introduction of cells into the cell chamber, the magnet can be moved to close proximity of the cell chamber to constrain the movement of the cells bound to the magnetic particles via application of magnetic forces thereto such that the other cells (the cells not bound to magnetic particles) can be removed from the cell chamber and collected in a non-magnetic fraction. The magnet can then be moved away from the proximity of the chamber, thus reducing or preferably eliminating the magnetic forces applied to the magnetic particle-bound cells such that those cells can also be removed from the cell chamber and transferred to a separate magnetic fraction. Further, in some embodiments, the device 602 can include various magnetic arrangements, electro-magnets, active fluid control, and cell chamber designs for optimal control of the magnetic forces during the separation process.

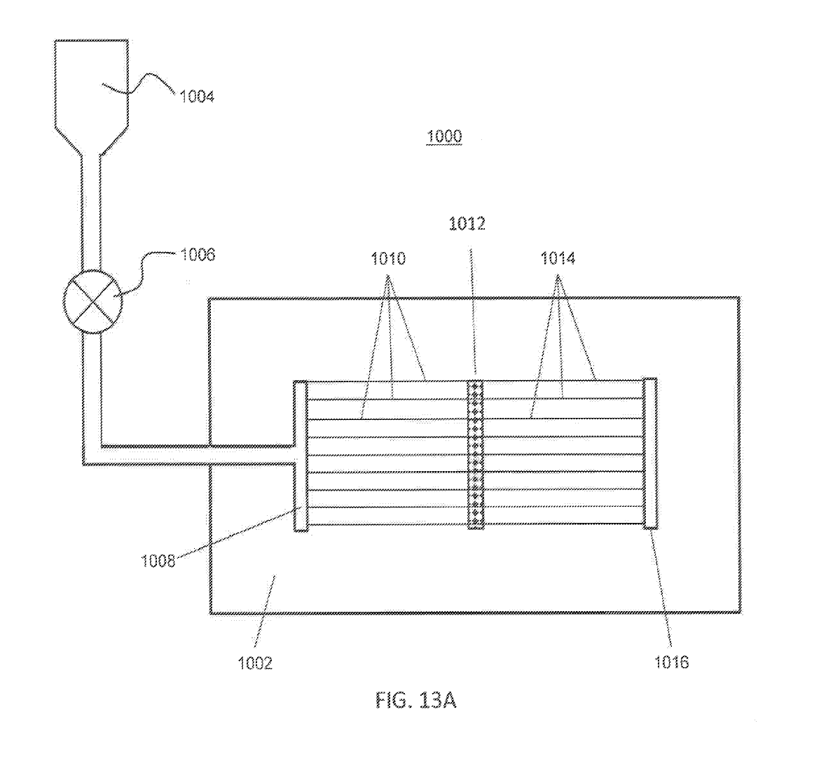

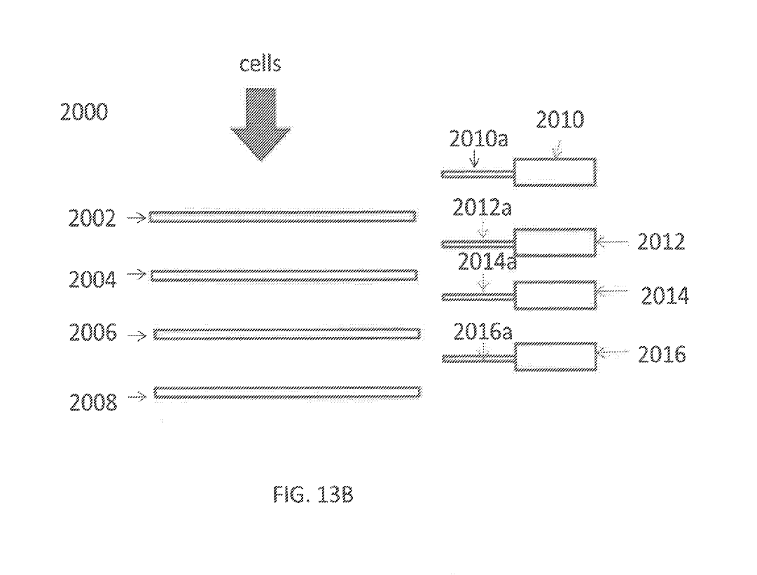

In some embodiments, a system for processing cells can include a microfluidic device, which can receive a sample containing cells from a reservoir and process those cells in a manner discussed above. By way of example, FIG. 13A schematically depicts such a system 1000 having a microfluidic device 1002 that is fluidly coupled to a sample reservoir 1004, which contains a plurality of cells and one or more agents contained in a liquid carrier. A pump 1006 facilitates the delivery of a flow of the liquid carrier together with the cells and agent(s) contained therein to the microfluidic device 1002. The microfluidic delivery device 1002 can in turn apportion the received liquid carrier containing cells and agent(s) into a plurality of samples and deliver those sample (typically concurrently) to parallel cell processing channels of a cell processing assembly that is fluidly coupled thereto.

More specifically, in this embodiment, the microfluidic device 1002 includes an input chamber 1008 that can receive a sample from the reservoir 1004. A plurality of microfluidic input channels 1010 carry different portions of the received sample in parallel to a porous membrane 1012. The porous membrane 1012 can include a plurality of pores having at least one cross-sectional dimension (and preferably a maximum cross-sectional dimension) less than the cells' diameters so as to facilitate the uptake of one or more agents present in the sample by the cells. A plurality of output channels 1014 carry the processed cells to an output chamber 1016 for collection.

With reference to FIG. 13B, in some embodiments, a system 2000 according to the present teachings for delivery of agent(s) to cells comprises a plurality of porous membranes 2002, 2004, 2006 and 2008 that are positioned in series so as to receive sequentially a plurality of cells. The porous membranes are similar to those discussed in connection with the other embodiments and include pores having a cross-sectional diameter that is sufficiently small so as to cause the cells passing through the pores to uptake one or more agent(s) to which the cells are exposed. By way of example, the pores of the membranes can have at least one cross-sectional dimension, and preferably a maximum cross-sectional dimension, less than about 40 microns, e.g., in a range of about 2 microns to about 40 microns, or in a range of about 2 microns to about 30 microns, or in a range of about 2 microns to about 20 microns, or in a range of about 2 microns to about 15 microns, or in a range of about 2 microns to about 10 microns.

The system further includes a plurality of reservoirs 2010, 2012, 2014, and 2016 (herein also referred to as liquid exchange modules) each of which contains one or more agent(s) (e.g., contained in a liquid carrier). Each reservoir is fluidly coupled to one of the porous membranes (e.g., via a plurality of nozzles 2010a, 2012a, 2014a, and 2016a) so as to expose the cells as to one or more agent(s) such that at least some of the cell uptake the agent(s) as they traverse through that membrane. In some cases, each reservoir contains a different agent such that as the cells pass sequentially through the porous membranes they would be exposed to different agents and hence sequentially uptake different agents.

The following example is provided for illustrative purposes to further elucidate various aspects of the invention, and is not intended to indicate necessarily optimal ways of practicing various aspects of the invention and/or optimal results that can be obtained.

### Example 1: Physical Delivery of CRISPR-Cas9 Ribonucleic Acid Protein to Human CD34+ Hematopoietic Stem and Progenitor Cells (HSPCs)

Cas9 protein holds tremendous potential not only for basic research, but for therapeutic applications for knocking genes out or even repairing genes. There are currently a handful approaches of delivery Cas9 into cells of interests which include, viral delivery of the Cas9 and guides, plasmid and mRNA delivery via lipid nanoparticles, or electroporation. These methods pose the risk of random integration of the Cas9/guide RNA sequences into the genome, which can lead to undesired consequences. These methodology also generate a high expression of Cas9 and gRNA proteins within the cell, which can tax the cell's protein synthesis mechanism with Cas9 or even increase the risk of off target and random cutting. Theoretically, only a single protein reaching the target genomic sequence is needed. In addition, plasmid DNA and mRNA have proven to be quite bulky and thus difficult for transient delivery into suspension cells like human hematopoietic stem and progenitor cells (HSPCs) and T-cells. In this example, the delivery of the Cas9 protein complexed with a sgRNA as a ribonucleoprotein complex (RNP, also referred to herein as a ribonuclear protein complex) is described, where the cargo is smaller, has a shorter lifespan, and poses no risk of random integration. Due to the limited intrinsic endocytosis activity, HSPCs require a physical direct delivery method like electroporation. But electroporation can be quite harsh to cells, leading to high cytotoxicity and reduced proliferation.

In this example, an alternative system, Transmembrane Internalization Assisted by Membrane Filtration (TRIAMF) is employed, which demonstrates high delivery of RNP complexes and high editing efficiency, with low cell toxicity, while allowing for scalability, and modularity for incorporation into other cell processing systems.

### Methods:

Setup of apparatus: systems such as those discussed above were employed. Stainless steel devices (single and 24-well manifold) and stainless disc membrane supports were custom-made in accordance with the present teachings. All other parts were purchased from McMaster Car. Track etched polycarbonate PVP coated membranes were purchased from Sterlitech (item number PCT8013100 (8 μm, thin), PCTF8013100 (8 μm thin, hydrophobic), PCT10013100 (10 μm, thin), and custom membranes with, 7 μm (thick), 8 μm (thick), 9 μm (thick), pore sizes with varying thicknesses). Cas9 protein was manufactured internally, and formulated in 20 mM HEPES, 150 mM KCl, 1% Sucrose, 1 mM TCEP at pH 7.5 or 20 mM TRIS-HCl at pH 8.0, 200 mM KCl, 10 mM MgCl₂, while the sgRNA molecules were synthesized and purchased from AxoLabs or TRILINK, and included a targeting domain complementary to a sequence of B2M (B2M targeting domain sequence: GGCCGAGAUGUCUCGCUCCG (SEQ ID NO: 6)). Frozen bone marrow CD34+ HSPCs were purchased from Lonza or AllCells. StemSpan SFEM II media (09655), CC100 (02690), were all purchased from Stem Cell Technologies. FITC conjugated B2M antibody (clone 2M2) and Human TruStain FcX were purchased from Biolegend.

### TRIAMF System Set Up

Silicon o-ring was placed, followed by stainless steel mesh, followed by polycarbonate membrane (shiny side up), then followed by Teflon washer, then the top was attached and tightened (24 well manifold via screws, single holders via wrenches). 3 mL syringes were then attached to the needles.

Prior to transfection, the manifold was calibrated by running 2 mL of sterilized DI water, followed by 1 mL of 70% ethanol and then followed by another 2 mL of sterile DI water at 5 psi.

### Human Hematopoietic Stem and Progenitor Cells Culture

Frozen bone marrow derived CD34+ HSPCs were purchased from Lonza or AllCells and thawed according to manufacturer's instruction. Cells were cultured in SFEM II supplemented with CC110 (StemCell Technologies), 0.75 uM StemRegenin 1 (StemCell Technologies), 50 nM UM171 (StemCell Technologies), 50 ng/mL human recombinant IL-6 (Peprotech), and PenStrep. Cells were cultured/expanded for 3-5 days before being used in experiments.

### RNP Delivery Via TRIAMF

Human hematopoietic stem and progenitor cells (0.5, 1, 2, 4, 5, or 10 million cells) were spun down and resuspended in 20 μL of SFEMII media. 200 μg of Cas9 was mixed with 40 μg of sgRNA targeting beta-2 microglobulin (“B2M”) and allowed to complex by incubating for 5 minutes at room temperature. The RNP mixture were mixed with the cells and allowed to incubate for 2 min at RT with a final volume of 50 μL. The mixture was then transferred into the bottom of the syringe connector with the manifold. Then the mixture is forced through the syringe and membrane via 5 psi of pressure.

The flow-through was allowed to rest for ˜2-5 minutes, before the membrane was washed with 1 mL of complete media. After rest, the cells were then supplemented with fresh SFEM II media supplemented with CC100/AHR antagonist ((S)-2-(6-(2-(1H-indol-3-yl)ethylamino-2-(5-fluoropyridin-3-yl)-9H-purin-9-yl)propan-1-ol; compound 157S according to WO2010/059401 or StemRegenin 1; 4-[2-[[2-(1-benzothiophen-3-yl)-9-propan-2-ylpurin-6-yl]amino]ethyl]phenol) at 1 million cells/mL. The cells were allowed to recover and expand ex vivo for 72 hours (hrs) before downstream analysis.

### Cell Lysis Prep for Next Generation Sequencing

Editing efficiency was determined via next generation sequencing by analyzing indel formation. Approximately 100 k cells were spun down, and cell lysate were extracted via ˜40 μL lysis buffer with proteinase K. 2 μL of the cell lysis extract was then used to amplify target sequence via primers (forward: ctctcaaaccacagggatcaca; reverse: ctccatcaccaagagagcctt and Platinum Taq polymerase (Clontech) and submitted for next generation sequencing.

### Antibody Staining to Detect B2M Knockdown

Functional editing was determined by assessing B2M knockdown via FITC conjugated anti-B2M (BioLegend) staining and FACS analysis. Cells were spun down and collected and stained with FITC conjugated B2M antibody with Human TruStain FcX for 30 minutes. The cells were washed twice and then analyzed with BD Fortessa Flow Cytometer with comparison to unstained cells to determine B2M knockdown percentage.

### Cell Recovery

Cell recovery was determined by running 500 μL of the cells 72 hour post transfection in the Beckman Coulter Vi-Cell to count viable cells. The cell recovery was determined by comparing the number of viable cells in a treated sample to an untreated cell sample size with the same number of cells seeded in the same volume via this equation:

$$Cell Recovery \% = \frac{( \# Viable cells of sample )}{average ( \# Viable cells from untreated cells )} .$$

### Results:

### Delivery of Cas9/RNP for B2M Knockdown in CD34+ Hematopoietic Progenitors Cells

A single membrane holder accommodating a sample volume of 50 microliters as well as a 24-well manifold, which could be used to pass samples through 24 membranes in parallel, were fabricated.

Using the 24 well manifold, the device was optimized for the targeted delivery of ribonucleoprotein complex (RNP) with a single guide RNA (sgRNA) complexed with Cas9 protein into human CD34+ hematopoietic stem cells derived from the bone marrow that were expanded for 6-7 days. For optimization, beta-2-microglobulin was targeted with the sgRNA 129 (targeting seq: GGCCGAGAUGUCUCGCUCCG) as a target that can be assayed by flow cytometry staining and next generation sequencing (NGS). B2M knockdown were then measured with antibody cell staining by flow cytometry, and next generation sequencing, and cell recovery was calculated by the ratio of the number of cells collected 48 hours post-delivery with an untreated sample via Vi-Cell.

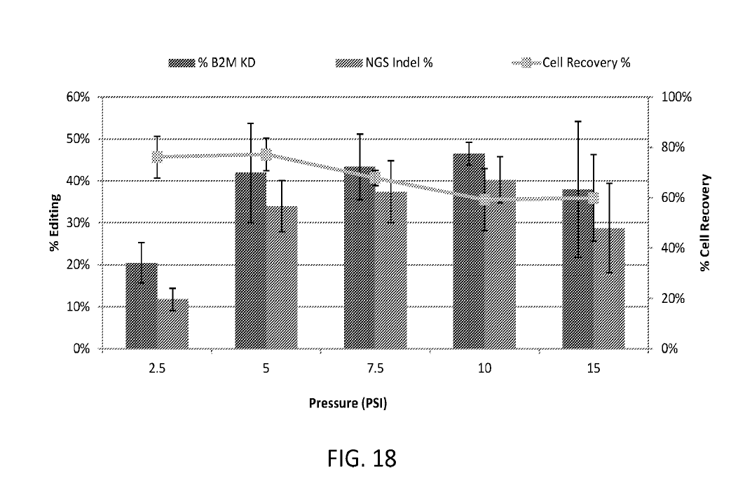

Various parameters were explored for optimization including: cell density, applied pressure, RNP concentration, membrane thickness, and membrane pore size. FIG. 18 depicts data indicating editing and cell recovery rates for delivery of Cas9/RNP complex at 200 μg of Cas9 into human hematopoietic stem cells at a concentration of 5 million cells per 50 μL as a function of applied pressure for passing the cells through a porous membrane in accordance with an embodiment of the invention. Using house nitrogen air, varying air pressures of 2.5, 5, 7.5, 10, and 15 PSI were tested. 2.5 psi was insufficient for effective delivery of RNPs with 20.5% B2M knockdown and 76% cell recovery. 5 PSI demonstrated 41.9% B2M knockdown and 77% cell recovery. Increasing the applied pressure to 7.5, 10, and 15 PSI did not increase the delivery efficacy, which remained at 40-45% with little decrease in the recovery of the cells.

200 μg of Cas9 (7.36 mg/mL) and 40 μg of sgRNA (5 mg/mL) were precomplexed together by mixing and incubating at RT for 5 min. Then the RNP complex was mixed with 0.5, 1, 2, 4, 5 or 10 million cells in a final volume of 50 μL of SFEM II. The RNP/cell mixture were then loaded onto the TRIAMF system (with 8 micron pore size and 7 micron thick membranes) and using a nitrogen air pressure of 5 psi, the sample was forced through the system and collected in a well plate. After 72 hrs, the cells were collected for next generation sequencing (NGS), FACS analysis, and Vi-Cell count to demonstrate successful gene editing by indel formation and functional B2M knockdown and cell recovery.

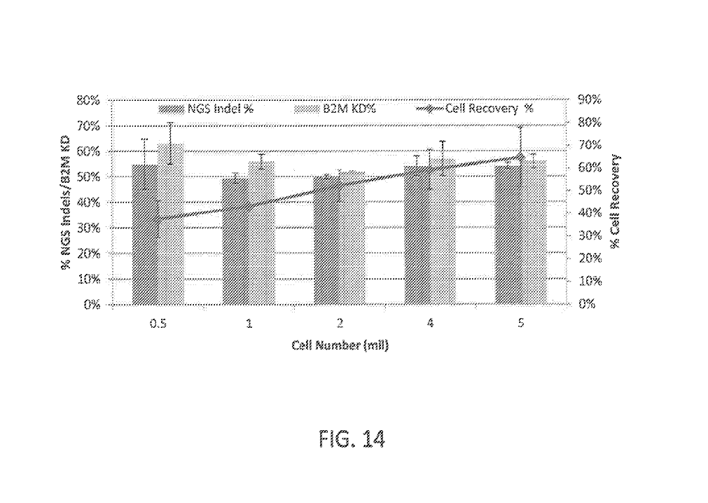

In FIG. 14, NGS indicates indel formation of 55%, 50%, 50%, 54%, and 54%. FACS analysis indicates B2M knockdown of 63%, 56%, 52%, 57%, and 56%, and a cell recovery of 38%, 43%, 52%, 60%, and 65% of 0.5, 1, 2, 4 and 5 million cells, respectively.

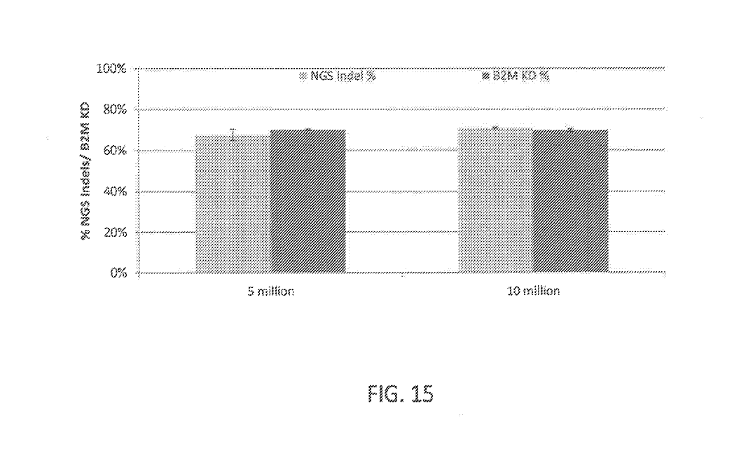

To determine the scalability of the system, cell concentrations of 5 million cells and 10 million cells per 50 μL were tested with 200 μg of Cas9 in 50 using the single manifold system. In FIG. 15, scaling up cell number from 5 to 10 million cells, TRIAMF achieved similar cutting efficiencies of 68% indel formation and 70% B2M knock down, and 71% indel formation and 70% B2M knockdown, respectively. These experiments demonstrate that the TRIAMF system is effective at delivering RNPs at varying cell numbers and densities, from 0.5 million up to 10 million cells without much change in editing efficiency. The cells were able to run through the system easily with no indications of clogging.

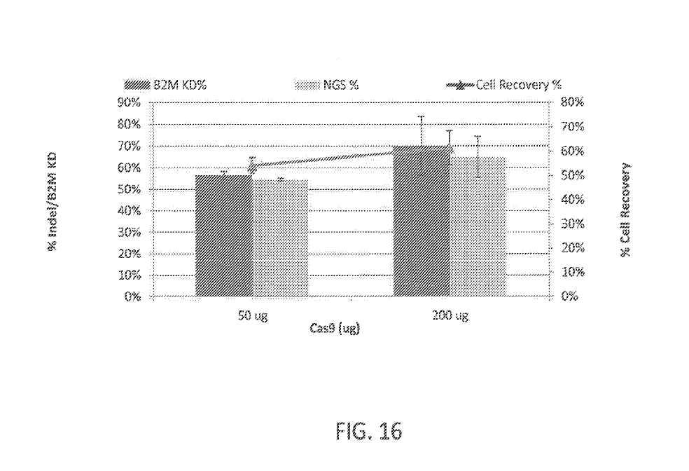

Different RNP concentrations were tested with the TRIAMF system (with 8 micron pore size and 7 micron thick membranes) at 50 μg or 200 μg of Cas9 with 10 μg or 40 of sgRNA, respectively. FIG. 16 demonstrates that 50 μg of Cas9, TRIAMF achieved 57% B2M knockdown, 55% indel formation, and 54% cell recovery, while at 200 μg of Cas9, 70% B2M knockdown, 65% indel formation, and 62% cell recovery were achieved. This experiment indicates the RNP concentration in the solution can be important for effective delivery, due to the need for passive diffusion of RNP to get into the cells after the pores are formed via membrane deformation. This indicates that there is a threshold of Cas9 concentration for most effective delivery via TRIAMF, with levels of editing achieved with both high and low-concentration RNP, but with higher concentrations of RNP leading to more effective editing.

Many of the membranes used had varying pore sizes and membrane thicknesses, as indicated in Table 1 below:

TABLE 1
Membrane Characteristics

| Pore size | Membrane thickness | Pore Density | |
|---|---|---|---|
| (micron) | (micron) | per cm² | Coating/surface |
| 7 | 19 (“thick”) | 4E5 | Hydrophilic (PVP) |
| 8 | 7 (“thin”) | 1E5 | Hydrophilic (PVP) |
| 8 | 18 (“thick”) | 2E5 | Hydrophilic (PVP) |
| 8 | 7 (“thin”) | 1E5 | Hydrophobic (“HP”) |
| 9 | 16 (“thick”) | 1E5 | Hydrophilic (PVP) |
| 10 | 10 (“thin”) | 1E5 | Hydrophilic (PVP) |

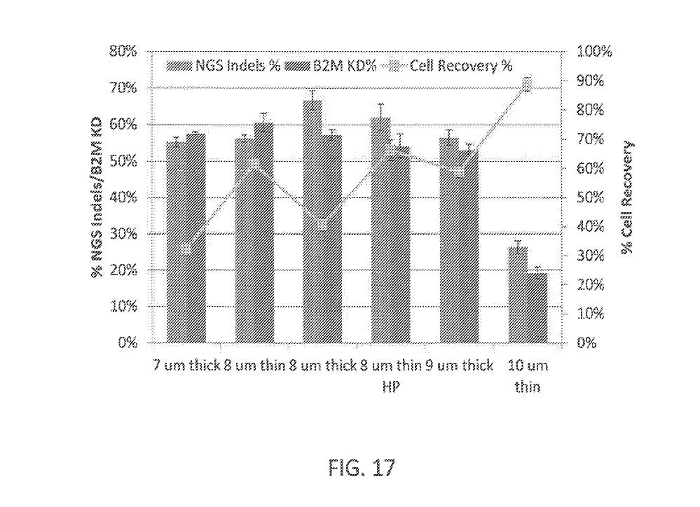

Different membrane parameters, including thickness and pore sizes were explored. Using the varying membranes, TRIAMF achieved optimal editing of HSPCs with 7 (micron pore size)/19 (micron thickness), 8/7, 8/18, 8/7 (hydrophobic), 9/16, and 10/10 membranes of 55% indel, 58% B2M knock down, and 32% cell recovery; 56% indel, 61% B2M knock down, and 62% cell recovery; 67% indel, 57% B2M knock down, and 41% cell recovery; 62% indel, 54% B2M knock down, and 66% cell recovery; 56% indel, 53% B2M knock down, and 59% cell recovery; 26% indel, 19% B2M knock down, and 89% cell recovery, respectively. FIG. 17 depicts TRIAMF delivery efficiency via indel % through NGS, B2M KD via FACS analysis and cell recovery through Vi-Cell of membranes per 50 of delivery volume with 5 million cells and RNP concentration of 200 μg of Cas9/50 sgRNA. In these experiments, no significant difference between the hydrophobic and hydrophilic membranes was observed. These data also indicate that given a specific pore size, for the thicker membranes, the cell recovery is reduced. The membrane pore sizes of 7, 8, or 9 microns provided the optimal efficacy, with cell likely to be directly flowing through 10 micron size membranes. This experiment demonstrates the optimal delivery of RNP to HSPCs is with the 8 micron pore size with 7 microns thick membranes, resulting in the optimal balance of editing and cell recovery.

Thus, in the above experiments, the optimal parameters include 5 million cells and 200 μg of Cas9 in 50 μL and passed through a 7 micron thick membrane with 8 micron pore sizes at an applied pressure of 5 PSI.

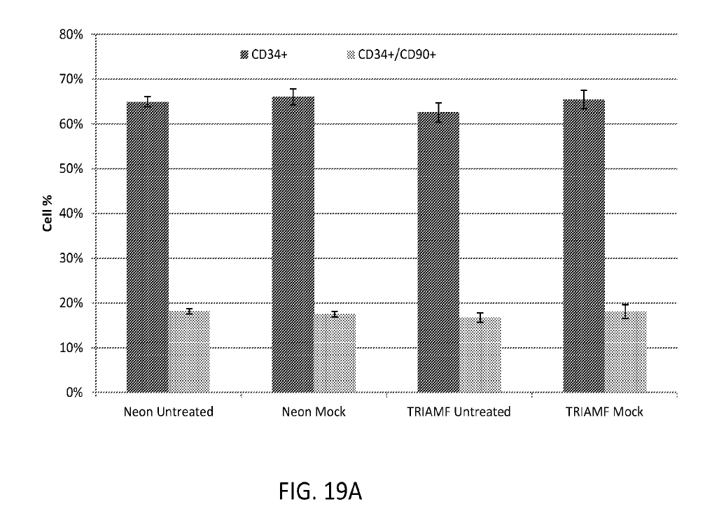

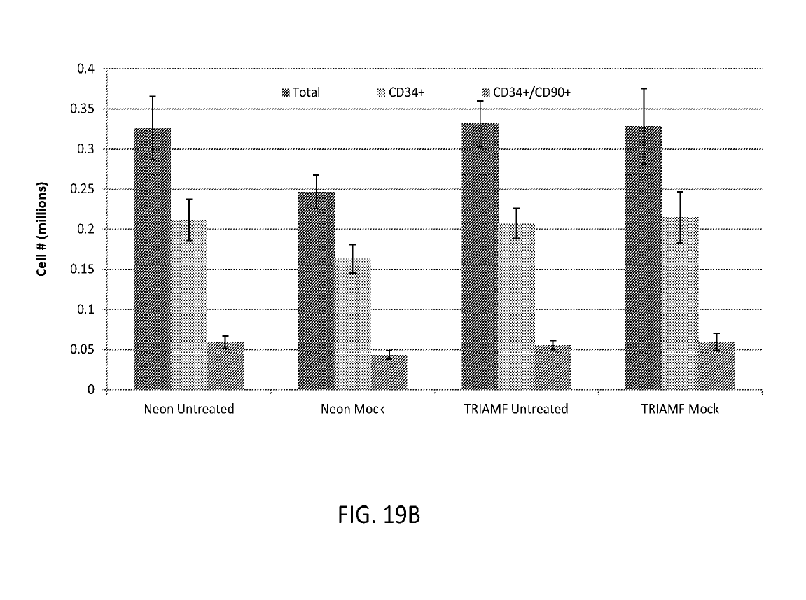

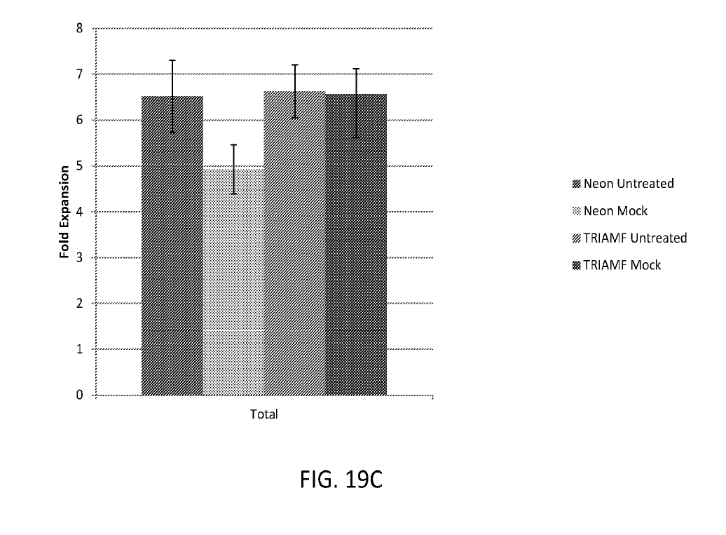

FIG. 19A depicts HSC surface markers by flow cytometry after 7 day expansion of 50 k/mL seeded cells 2 days after mock treatment via TRIAMF or neon electroporation. Comparison of CD34 and CD90 percentages by flow cytometry staining are also shown. FIG. 19B depicts HSC cell count after 7 day expansion of 50 k/mL seeded cells 2 days after mock treatment of TRIAMF or neon electroporation. Comparison of total, CD34+, and CD34+/CD90+ cell counts via Vi-Cell with flow cytometry staining is also depicted. FIG. 19C depicts HSC fold expansion after 7 day expansion of 50 k/mL seeded cells 2 days after mock treatment via TRIAMF or neon electroporation. The data indicates that electroporation significantly affects cell's ability to expand, while TRIAMF treatment allow the cells to expand normally.

These data demonstrates successful Cas9 RNP complex delivery into human CD34+ HSPCs using the TRIAMF system, which leads to effective gene editing. This can then be further applied to gene editing therapies for various genetic diseases including, for example, sickle cell anemia, thalassemia, and others.

### Example 2: HbF Upregulation Via HBG Knockdown Via Delivery of Cas9/RNP

Sickle cell disease is a debilitating multi-organ systemic disease with over 300,000 births a year globally, with over 70% in Sub-Saharan Africa. Sickle cell disease is characterized as the first molecular disease, where patients have a single nucleotide mutation from Adenine to Thymine, which causes a change in amino acid of Glutamic Acid to Valine. The change in mutation leads to the polymerization of sickle adult beta hemoglobin (HBB) under hypoxic conditions, leading to the sickle deformity of the red blood cells (RBC). The sickled RBCs has a deformed nature reducing its ability to travel through the blood vessels, leading to stroke, nephropathy, acute chest syndrome, infections, pain crises, and anemia. The current standard of care is the administration of hydroxurea, but currently 14-40% of the patients do not respond to the treatment, or require lifelong treatment. Hydroxyurea can have severe side effects including a suppressed immune system. Because of immunosuppressive effect of hydroxyurea, this treatment can cause high rate of death from infections in countries with limited healthcare and sanitation. Currently the only cure for sickle cell disease, is through allogenic bone marrow transplantation (BMT). BMT is limited to developing countries due to resources and the ability to screen for matched donors, which only fit about 20%.

### Results:

Similar method and system step-up as those discussed above in connection with Example 1 were utilized. In particular, a single membrane holder accommodating a sample volume of 50 microliters as well as a 24-well manifold, which could be used to pass samples through 24 membranes in parallel, were fabricated.

Using the 24 well manifold, the device was optimized for the targeted delivery of ribonucleoprotein complex (RNP) with a single guide RNA (sgRNA) complexed with Cas9 protein into human CD34+ hematopoietic stem cells derived from the bone marrow that were expanded for 6-7 days. For optimization, beta-2-microglobulin was targeted with the sgRNA 129 (targeting seq: GGCCGAGAUGUCUCGCUCCG) as a target that can be assayed by flow cytometry staining and next generation sequencing (NGS). B2M knockdown were then measured with antibody cell staining by flow cytometry, and next generation sequencing, and cell recovery was calculated by the ratio of the number of cells collected 48 hours post-delivery with an untreated sample via Vi-Cell.

The present teachings (herein also referred to as TRIAMF for brevity) were applied for the delivery of RNPs consisting of guides targeting the HBG locust to recapitulate the genotype of a form of hereditary persistent fetal hemoglobin found in many patients. Using a previously reported RNA targeting a globin locus sequence (Traxler et al., Nat. Med., 22(987-990); 2016; doi:10.1038/nm.4170, which is herein incorporated by reference in its entirety) in conjunction with the optimized protocol and apparatus of the present invention, HSCs expanded for 3 days ex vivo were transfected with RNPs with a gRNA targeting HBG, and an aliquot were then directly differentiated into erythrocytes using a two-step method, which included first inducing and expanding erythroid progenitors for 10 days and then maturing the erythroid progenitor cells to erythrocytes for 11 days. The rest of the cells were collected after 2 days for NGS and qPCR to determine indel frequency and pattern. Through a combination of next generation sequencing (NGS) and quantitative PCR (qPCR), editing efficiency at the target site was determined to be a total average of 63% total editing.

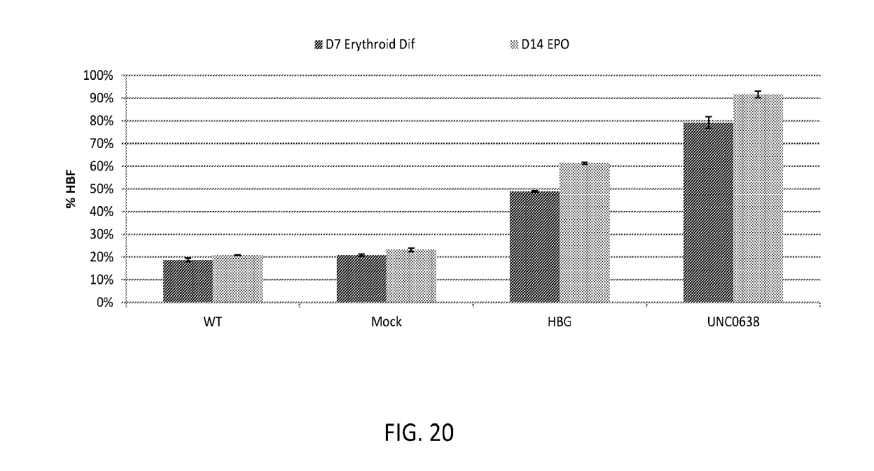

FIG. 20 depicts percentage of HbF cells at D7 and D14 of erythroid differentiation protocol of WT, mock treated, HBG edited, and UNC0638 (Blood. 2015, July 30:126(5):665-72) positive control treated cells. Cells were analyzed by fixation and staining of CD235a,CD71, and HbF. The cells were collected on days 7 and 14 and stained with CD235a, CD71, and fetal hemoglobin, and analyzed with flow cytometry. As shown in FIG. 20, comparing the HbF levels in untreated cells, mock treated cells (i.e., the cells that were put through the membranes without being exposed to an agent, such as Cas9/RNP), edited cells, and a positive control with UNC0638, 21%, 23%, 60%, and 92% of the cells expressed HbF, respectively.

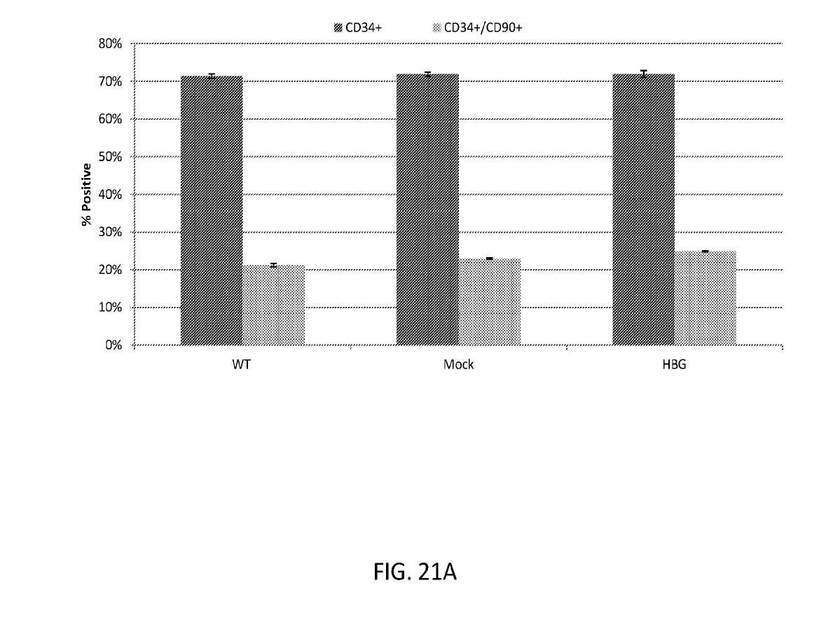

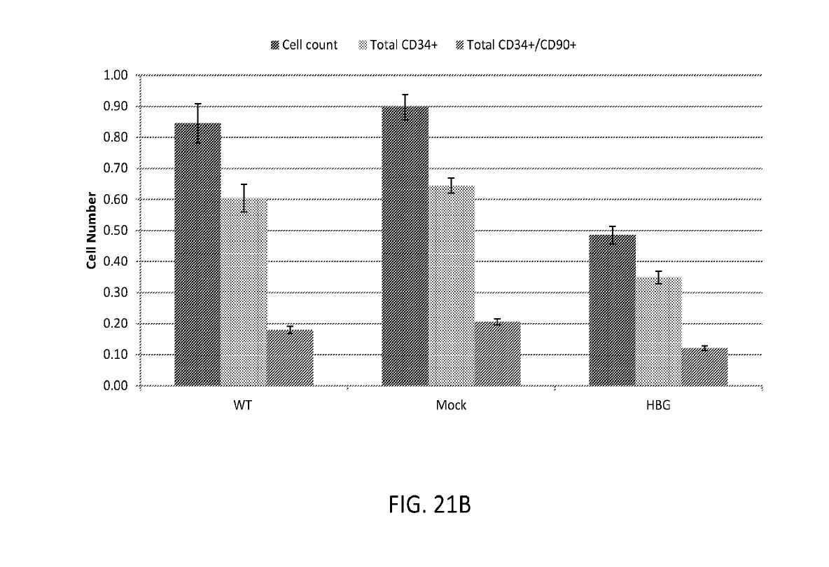

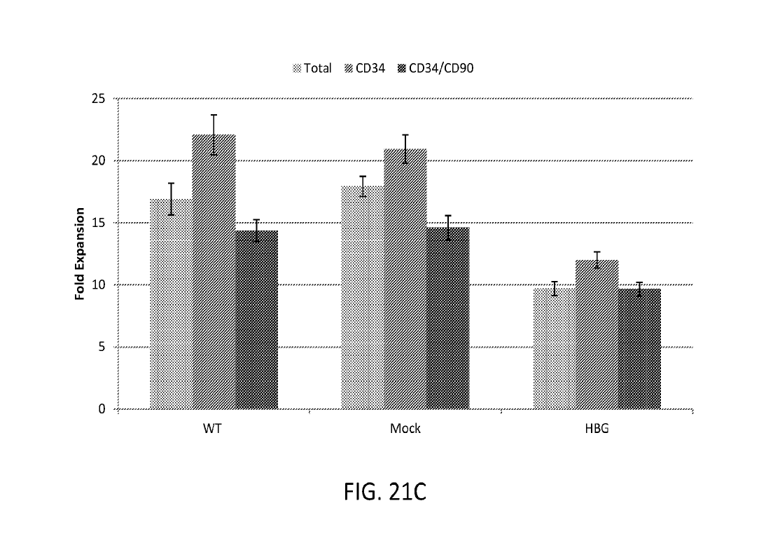

FIG. 21A depicts HSC surface markers by flow cytometry after 7 day expansion of 50 k/mL seeded cells 2 days after recovery of WT cells, mock TRIAMF treated cells, and HBG edited cells via TRIAMF. Comparison of CD34 and CD90 percentages by flow cytometry staining is also shown. FIG. 21B depicts HSC cell count after 7 day expansion of 50 k/mL seeded cells 2 days after recovery of WT cells, mock TRIAMF treated cells, and HBG edited cell via TRIAMF. Comparison of total, CD34+, and CD34+/CD90+ cell counts via Vi-Cell with flow cytometry staining is also shown. FIG. 21C depicts HSC fold expansion after 7 day expansion of 50 k/mL seeded cells 2 days after recovery of WT cells, mock TRIAMF treated cells, and HBG edited cell via TRIAMF.

After letting the transfected cells to rest for 2 days, the cells were counted and 50 k cells were expanded for 7 days to determine the cells' expansion capability after transfection according to the present teachings. After 7 days, the CD34+ and CD34+/CD90+ population seemed to remain the same at 70% and 20%, respectively. As shown in FIGS. 21A-21C, the observation of cell count and fold expansion from seeding showed that the HSCs treated by transfection methods of the present teachings were comparable with the untreated cells, with no significant changes. But for the cells that were treated with the HBG targeting RNPs, there was a significant reduction of cell expansion, potentially due to toxicity caused by DNA damage from the double strand breaks.

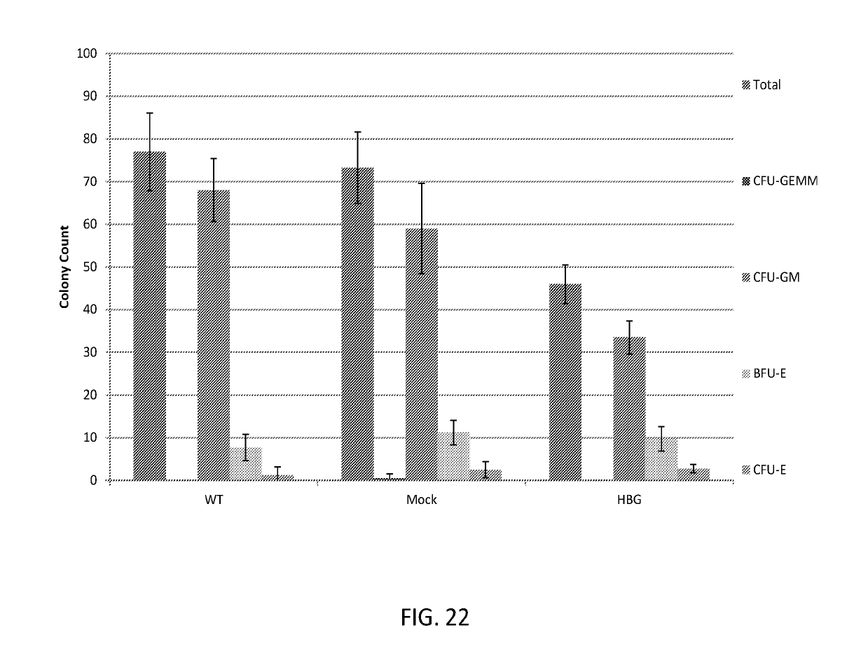

FIG. 22 depicts the result of colony forming assay to determine the TRIAMF treated HSC's ability to commit to different lineages. Colony counts of different hematapoeitic lineages after 14 days of growth on methocult are shown (plates were imaged and analyzed with StemVision). A colony forming assay was also conducted on the HSCs 2 days after transfection to confirm that the TRIAMF system did not alter the cells multipotency and ability to differentiate. The HSCs that were untreated, mock treated through TRIAMF, and HBG edited with TRIAMF gave rise to a total of 77, 73, and 46 average total colonies, respectively (as shown in FIG. 22). These colonies also gave rise to a similar pattern of erythrocytes (BFU-E), granulocytes and macrophages (GM), macrophages (M), and granulocytes, erythrocytes, monocytes, and megakaryocytes (GEMM) multicellular colonies. The cells that had the RNPs with the sgRNA targeting HBG delivered via TRIAMF, demonstrated a large drop in colonies formed, which can be the result of toxicity from DNA damage from Cas9 mediated double strand breaks.

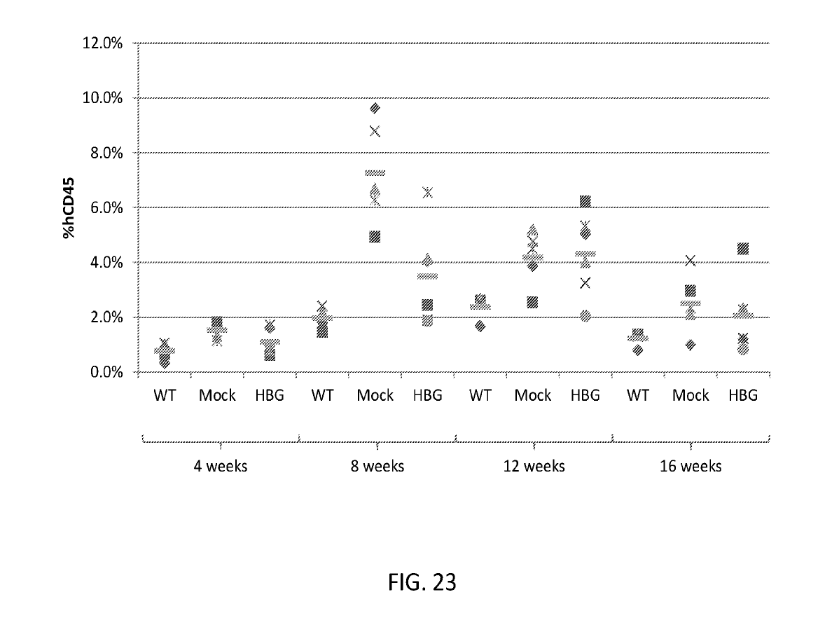

FIG. 23 presents data indicating that TRIAMF edited HSPCs display long-term reconstitution in NSG mice. 700 k TRIAMF treated cells were tail vein injected into each mouse (WT: N=4, Mock: N=5, HBG: N=6). Peripheral blood was collected every 4 weeks and stained for anti-human and anti-mouse CD45 and human CD45. To fully prove that the modified HSCs were functional, wild type (WT), Mock TRIAMF, and HBG edited via TRIAMF cells were transplanted into sub-lethally irradiated NOD/SCID gamma (NSG) mice. All cells transplanted into the mice demonstrated human engraftment in the peripheral blood at 16 weeks, with an average of 1.2%, 2.5%, and 2.1% hCD45+ in the WT, mock TRIAMF, and HBG edited via TRIAMF, respectively (shown in FIG. 23).

To explore the effects of TRIAMF on HSCs, mock transfections (i.e., in absence of any cargo such as Cas9/RNP) using electroporation and TRIAMF systems were compared. Using the Neon electroporation system marketed by Thermofisher and the TRIAMF system described above, cells were run through either system without any agent and the cells' ability to expand for 7 days was compared for the two systems. Although the cells treated by both systems demonstrated similar population pattern of 65% CD34 positive cells and about 17% CD34/CD90 double positive cells, the cell expansion number and fold expansion of TRIAMF treated cells stayed the same at 0.33 million total, 0.21 million CD34+, and 0.06 CD34+/CD90+ cells. But the Neon mock treated cells dropped at 0.24 million total, 0.16 million CD34+, and 0.04 CD34+/CD90+ cells. After 7 days, the fold expansion of the WT cells and TRIAMF mock treated cells were at 6.5 fold, while the fold expansion of the mock electroporated cells was only at 5 fold (shown in FIGS. 19A-19C).

### Methods:

Material: Stainless steel devices (single and 24 well manifold) and stainless steel disc membrane supports were custom-made in accordance with the present teachings. All other parts were purchased from McMaster Car. Track etched polycarbonate PVP coated membranes were purchased from Sterlitech (item number PCT8013100 (8 μm, thin). Cas9 protein was manufactured internally, and formulated in 20 mM HEPES, 150 mM KCl, 1% Sucrose, 1 mM TCEP at pH 7.5 or 20 mM TRIS-HCl at pH 8.0, 200 mM KCl, 10 mM MgCl2), while the sgRNA molecules were synthesized and purchased from AxoLabs, and included a targeting domain complementary to a sequence of HBG (CUUGUCAAGGCUAUUGGUCA). Frozen bone marrow CD34+ HSPCs were purchased from Lonza or AllCells. StemSpan SFEM II media (09655), CC100 (02690), were all purchased from Stem Cell Technologies. FITC conjugated B2M antibody (clone 2M2) and Human TruStain FcX were purchased from Biolegend.

### TRIAMF System Set Up

Silicon O-ring was placed, followed by stainless steel mesh, followed by polycarbonate membrane (shiny side up), followed by Teflon washer, the top was attached and tightened (24 well manifold via screws, single holders via wrenches). 3 mL syringes were then attached to the needles.

Prior to transfection, the manifold was calibrated by running 2 mL of sterilized DI water, followed by 1 mL of 70% ethanol and then followed by another 2 mL of sterile DI water at 5 psi.

### Human Hematopoietic Stem and Progenitor Cells Culture

Frozen bone marrow derived CD34+ HSPCs were purchased from Lonza or AllCells and thawed according to manufacturer's instruction. Cells were cultured in SFEM II supplemented with CC110 (StemCell Technologies), 0.75 uM StemRegenin 1 (StemCell Technologies), 50 nM UM171 (StemCell Technologies), 50 ng/mL human recombinant IL-6 (Peprotech), and PenStrep. Cells were cultured/expanded for 3-5 days before being used in experiments.

### RNP Delivery Via TRIAMF

Human hematopoietic stem and progenitor cells (5 million cells) were spun down and resuspended in 20 μL of SFEMII media. 200 μg of Cas9 was mixed with 40 μg of sgRNA targeting HBG and allowed to complex by incubating for 5 minutes at room temperature. The RNP mixture were mixed with the cells and allowed to incubate for 2 min at room temperature (RT) with a final volume of 50 μL. The mixture was then transferred into the bottom of the syringe connector with the manifold. Then the mixture was forced through the syringe and membrane via 5 psi of pressure.

The flow-through is allowed to rest for ˜2-5 minutes, before the membrane is washed with 1 mL of complete media. After rest, the cells were then supplemented with fresh media. The cells were allowed to recover and expand ex vivo for 72 hours (hrs) before downstream analysis.

### Cell Lysis Prep for Next Generation Sequencing

Editing efficiency was determined via next generation sequencing by analyzing indel formation. Approximately 100 k cells were spun down, and cell lysate was extracted via ˜40 μL lysis buffer with proteinase K. 2 μL of the cell lysis extract were then used to amplify target sequence via primers (HBG1 forward: cgctgaaactgtggctttatagaaatt; HBG1 reverse: ggcgtctggactaggagcttattg; HBG2 forward: gcactgaaactgttgctttataggat; HBG2 reverse: ggcgtctggactaggagcttattg and Platinum Taq polymerase (Clontech), and then submitted for next generation sequencing.

### OCR Detection of Large HbF Deletions

To detect large deletions, a primer and probe set was designed to detect the amplification of an HBG1 promoter specific sequence. HBG Fwd: ACGGATAAGTAGATATTGAGGTAAGC, HBG Rev: GTCTCTTTCAGTTAGCAGTGG, and TaqMan probe (FAM): ACTGCGCTGAAACTGTGGCTTTATAG. TaqMan qPCR was performed on genomic DNA samples from CD34+ cells using the Universal TaqMan Mix (Thermo Fisher Scientific). Average ΔCT values were calculated by subtracting average CT value of HbG minus average CT of RPPH1 (Thermo Fisher Scientific), for copy-number reference for each gDNA samples and the ΔΔ Ct values were calculated by subtracting ΔCT value of edited sample minus average mock ΔCT. Fold was calculated by taking (2\hat{ }-ΔΔCT).

### Cell Recovery

Cell recovery was determined by running 500 μL of the cells 72 hour post transfection in the Beckman Coulter Vi-Cell to count viable cells. The cell recovery was determined by comparing to an untreated cell sample size with the same number of cells seeded in the same volume via this equation:

$$Cell Recovery \% = \frac{( \# Viable cells of sample )}{average ( \# Viable cells from untreated cells )} .$$

### Erythroid Differentiation and HbF Detection

Erythroid differentiation was done following StemCell Technologies' two phase method. Briefly, directly after RNP delivery via TRIAMF, 10 k cells/mL were cultured in SFEM II supplemented with Erythroid Expansion Supplement (02692) and Pen Strep for 10 days. After 10 days, the cells were washed once with PBS, and then cultured in SFEM II supplemented with 3% human AB serum, 3 U/mL of recombinant human EPO, and Pen Strep. 0.5 μM of compound UNC0638 was used as a positive control to induce high levels of HbF. Cells were cultured for an additional 11 days. After 4 and 11 days, cells were collected, fixed, permeabilized, and stained with APC conjugated anti-CD71 (BioLegend), FITC conjugated anti −GlyA (CD235a) (BioLegend), and PE conjugated anti-HbF (Invitrogen, HBF-1 clone).

### CFU Assay

Colony forming assay was conducted with Methocult Optimum (Stem Cell Technologies) according to manufacturer's instructions. Briefly, 2 days after RNP delivery via TRIAMF, the cells were counted, diluted, and plated at 300 cells per 6-well well. After 14 days, colonies were counted and scored automatically with the StemVision system (StemCell Technologies).

### In-Vivo Engraftment

Cells were recovered for 2 days after thawing then RNP containing the HBG guide was delivered by TRIAMF. After 1 day of recovery, the cells were washed once with PBS and then counted. 3 groups were prepared: wildtype, mock, and edited. Using tail IV injection, 700 k cells from each group were injected to each 6-8 week old NSG mice (Jackson Laboratory). At16, 20, and 24 weeks, peripheral blood samples were collected via tail snip or retro-orbital bleed, RBCs were lysed with ACK buffer, and then stained with anti-human and mouse CD45 antibodies to quantify human cell engraftment and contribution.

At week 24 after transplantation, mice were euthanized, mouse bones (2×femur, 2× tibia, 2× iliac crest, and spine) were collected and crushed using mortar and pestle. RBCs were then lysed with ACK buffer. A fraction of the cells were stained human CD45, mouse CD45, CD3, CD19, CD33, and CD71 for lineage markers. Another fraction of cells were stained for human CD45, mouse CD45, CD34, and CD90 for hematopoietic stem cell markers. Cells were also FACS sorted to isolate human CD34 cells that were collected for NGS for editing and indel analysis and for erythroid differentiation for detecting HbF upregulation.

### Example 3: Physical Delivery Nucleic Acid and Peptides to Human CD34+ Hematopoietic Stem and Progenitor Cells (HSPCs)

Due to the limited endocytosis activity, HSPCs require a physical direct delivery method like electroporation. But electroporation can be quite harsh to cells, leading to high cytotoxicity and reduced proliferation.

The methods and system of the present teachings (which are herein also referred to as Transmembrane Internalization Assisted by Membrane Filtration (TRIAMF)) can be utilized for effective delivery of different nucleotides and peptides into cells, while allowing for scalability, and modularity for incorporation into other cell processing systems. This allows for introduction of nucleic acids or peptides into HSCs for therapeutic applications.

### Methods:

An apparatus such as that depicted in FIGS. 4B, 5A was employed. Silicon O-ring was placed, followed by stainless steel mesh, followed by polycarbonate membrane (shiny side up), then followed by Teflon washer, then the top was attached and tightened (24 well manifold via screws, single holders via wrenches). 3 mL syringes were then attached to the needles.

Prior to transfection, the manifold was calibrated by running 2 mL of sterilized DI water, followed by 1 mL of 70% ethanol and then followed by another 2 mL of sterile DI water at 5 psi.

Human hematopoietic stem and progenitor cells (4 million cells) were spun down and re-suspended in 20 μL of SFEMII media. The mRNA (60 ug), minicircle DNA (5, 10, 20 μg), or peptide tagged with CY5 (30 ug) were mixed with the cells and allowed to incubate for 2 min at RT with a final volume of 50 μL. The mixture was then transferred into the bottom of the syringe connector with the manifold. Then the mixture was forced through the syringe and membrane via 5 psi of pressure.

The flow-through was allowed to rest for ˜2-5 minutes, before the membrane was washed with 1 mL of complete media. After rest, the cells were then supplemented with fresh HSC media at 1 million cells/mL. The cells were allowed to recover and expand ex vivo before downstream analysis. Delivery efficacy was analyzed at various time points using flow cytometry to detect GFP or CY5.

Cell recovery was determined by running 500 μL of the cells 24 or 48 hour post transfection in the Beckman Coulter Vi-Cell to count viable cells. The cell recovery was determined by comparing to an untreated cell sample size with the same number of cells seeded in the same volume via this equation: Cell Recovery %=((# Viable cells of sample))/(average(# Viable cells from untreated cells)).

### Material:

The single and 24 well manifold and the stainless steel disc membrane supports were custom made. All other parts were purchased from McMaster Car. Track etched polycarbonate PVP coated membranes were purchased from Sterlitech (item number PCT8013100 (8 um, thin)). Frozen bone marrow CD34+ HSPCs were purchased from Lonza or AllCells. mRNA and Cy-5 peptides (MW:2637) were manufactured internally, while the minicircle DNA encoding GFP (MN601MC) was purchased from System Biosciences.

Results: mRNA Delivery

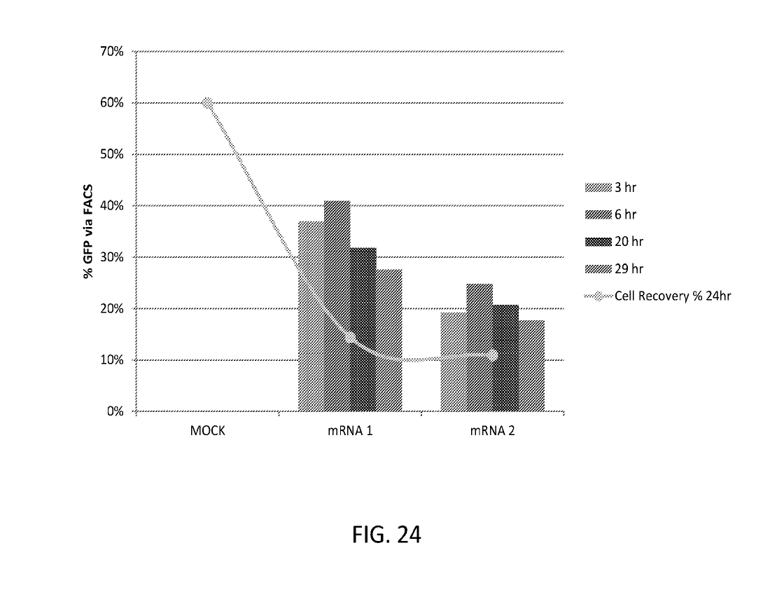

FIG. 24 depicts the results of flow cytometry analysis of GFP expression in HSCs at different time points after delivery of 60 μg of mRNA in accordance with an embodiment of the present invention, along with percentage cell recovery after 24 hours. The use of TRIAMF to deliver mRNA coded for GFP into HSCs was demonstrated. Using the same 8 μm pore size membranes, 60 μg of mRNA was delivered to 4 million cells. 6 hours after transfection, FACS was run on the cells (FIG. 24). A41% efficacy was observed with mRNA 1, and 25% efficacy was observed with mRNA 2, with a cell recovery of about 10-15% 24 hours after transfection, while the mock treated cells had 60% cell recovery. Although the cell recovery of the mock treated cells was at 60%, the cell recovery for mRNA treated cells was significantly lower, potentially due to foreign nucleic acid response of the HSCs.

### Minicircle Delivery

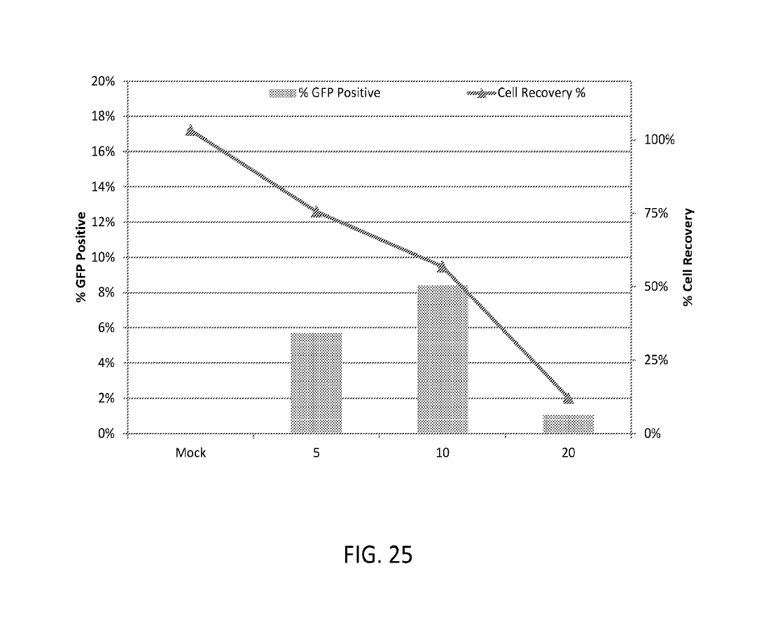

FIG. 25 depicts the results of flow cytometry analysis of GFP expression in HSCs at different time points after delivery of minicircle DNA at 5, 10 and 20 μg of DNA in accordance with an embodiment of the present invention, along with percentage cell recovery after 24 hours. The use of TRIAMF to deliver minicircle DNA encoded with GFP into HSCs was demonstrated. Using the 8 μm pore size membranes, 5, 10, or 20 ups of plasmid DNA were delivered into 4 million cells. After 48 hours, the cells were analyzed by flow cytometry (FIG. 25). For 5, 10 and 20 μg, the GFP expressions were 5.7%, 8.4%, and 1% respectively, with a cell recovery percentage of 75%, 57%, and 12%, respectively. Although the cell recovery of the mock treated the cells was at 60%, the cell recovery for the minicircle treated cells was significantly lower, potentially due to foreign nucleic acid response of the HSCs.

### Peptide Delivery

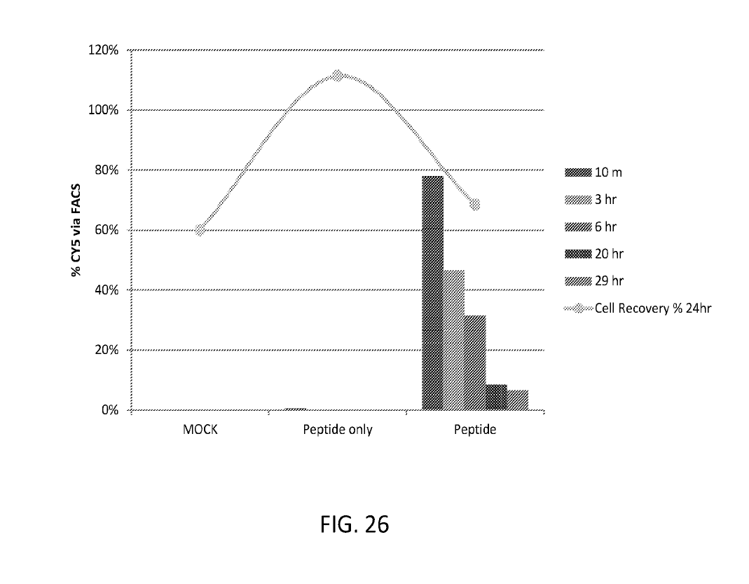

FIG. 26 depicts the results of flow cytometry analysis of Cy5 signal in HSCs at different time points after delivery of 30 μg of CY5-tagged peptides, along with percentage cell recovery after 24 hours. Using the TRIAMF system and 8 micron pore size membranes, 30 μg of Cy-5 tagged peptides was delivered to 4 million HSCs. After 10 minutes and a few washes (FIG. 26), 78% of the cells were observed to internalize the peptides, but after 24 hours, a steady drop was observed as the peptide was potentially digested. At 24 hours the cell recovery % of the cells was at 68% which is comparable with the Mock, which was at 60%.

Those having ordinary skill in the art will appreciate that various changes can be made to the above embodiments without departing from the scope of the invention. All publications referenced herein are incorporated by reference in their entirety.

1. A method of cell processing, comprising:
passing a plurality of cells through one or more pores of a membrane comprising a plurality of pores while exposing the cells to an agent so as to cause a change in the cells, thereby allowing said agent to enter at least one of the cells;
wherein each of said pores extends from an input opening to an output opening and has a maximum cross-sectional dimension in a range of about 7 microns to about 9 microns.

2. The method of claim 1, wherein said maximum cross-sectional dimension is in a range of about 8 microns to about 9 microns.

3. (canceled)

4. The method of claim 1, wherein said circulatory cells are selected from the group consisting of CD34+ cells, progenitor cells, induced pluripotent stem cells (iPSCs), hematopoietic stem and progenitor cells (HSPCs), immune effector cells, CD3+ cells, T cells and NK cells.

5. (canceled)

6. The method of claim 1, wherein said circulatory cells are any of engineered to express a chimeric antigen receptor (CAR) or capable of being engineered to express a chimeric antigen receptor (CAR).

7. (canceled)

8. The method of claim 1, wherein said pores have a length in a range of about 7 microns to about 10 microns.

9. (canceled)

10. The method of claim 1, wherein said membrane has an active surface area in a range of about 7 mm² to about 80 mm².

11. The method of claim 1, wherein said membrane has a surface pore density in a range of about 1×10⁵ to about 2×10⁶ pores/cm².

12. The method of claim 1, wherein the cells and the agent are disposed within a liquid carrier and said liquid carrier is pushed through said pores via application of a pressure thereto at a rate in a range of about 10 to about 20 mL (milliliters) per minute.

13. The method of claim 12, wherein a concentration of said cells within said liquid carrier is in a range of about 10,000 to about 200,000 cells per microliter.

14. (canceled)

15. (canceled)

16. The method of claim 12, wherein said liquid carrier comprises any of water, a basal cell culture medium, a serum free medium, HSC brew, one or more cytokines, one or more growth factors, one or more viability enhancers, polyethylene glycol (PEG), a detergent, a membrane stabilizer and a combination thereof, and optionally wherein the one or more viability enhancers are any of UM171, SR1, ((S)-2-(6-(2-(1H-indol-3-yl)ethylamino)-2-(5-fluoropyridin-3-yl)-9H-purin-9-yl)propan-1-ol, and combinations thereof.

17. (canceled)

18. The method of claim 1, wherein said change is a transient change comprising a change in permeability of the cells' membranes.

19. (canceled)

20. The method of claim 1, wherein said pores of the membrane are at least partially coated with polyvinylpyrrolidone.

21. The method of claim 1, wherein said agent comprises any of a deoxyribonucleic acid (DNA), an ribonucleic acid (RNA), a plasmid, a ribonuclear protein complex (RNP), a protein, a peptide, a lipid, a polysaccharide, an oligosaccharide, an antisense oligonucleotide, an aptamer, a nanoparticle, a dye, a template nucleic acid, a non-membrane permeable compound and combinations thereof.

22. The method of claim 1, wherein the agent is a gene editing system.

23. The method of claim 22, wherein the gene editing system is a CRISPR gene editing system, ZFN gene editing system, TALEN gene editing system, meganuclease gene editing system or Cre recombinase gene editing system.

24. (canceled)

25. The method of claim 23, wherein the CRISPR gene editing system comprises one or more RNP.

26. The method of claim 25, wherein said RNP is a Cas9-gRNA complex.

27. (canceled)

28. (canceled)

29. The method of claim 1, wherein said agent is any of electrically charged and electrically neutral.

30. (canceled)

31. The method of claim 1, wherein said agent has a molecular weight greater than about 2 kDa.

32. The method of claim 1, wherein said membrane comprises a polymeric material.

33. The method of claim 32, wherein said polymeric material is selected from the group consisting of polycarbonate, polytetrafluoroethylene (PTFE), polystyrene, polyvinylidene fluoride (PVDF), polyethylene terephthalate (PET), poly methyl methacrylate (PMMA), polypropylene (PP), polyimide (PI), cyclic olefin copolymer (COC), cyclo olefin polymer (COP), polyester, and polydimethylsiloxane (PDMS).

34. The method of claim 32, wherein said pores are formed in said polymeric material via any of ion track etching, laser drilling, plasma etching, or photolithography.

35. The method of claim 1, wherein said membrane comprises any of a semiconductor, ceramic, or a metal.

36. The method of claim 1, wherein said pores have a substantially uniform cross-sectional area along a length of each of said pores.

37. (canceled)

38. The method of claim 1, wherein said pores have a any of a regular and an irregular cross-sectional shape.

39. (canceled)

40. The method of claim 38, wherein said regular cross-sectional shape is any of a circular, an oval and a polygonal shape.

41. (canceled)

42. The method of claim 1, wherein said pores have any of hydrophobic and hydrophilic inner surfaces.

43. (canceled)

44. The method of claim 1, further comprising selecting said cells from a collection of heterogeneous cells prior to the step of passing the cells through the membrane.

45. The method of claim 44, wherein said selected cells are selected from the group consisting of CD34+, hematopoietic stem cells, hematopoietic progenitor cells, hematopoietic stem and progenitor cells (HSPCs), immune effector cells, CD3+ cells, T cells and NK cells

46. The method of claim 44, wherein said selected cells are any of engineered to express a chimeric antigen receptor (CAR) and capable of being engineered to express a chimeric antigen receptor (CAR), and optionally wherein said selected cells are T cells or NK cells.

47. (canceled)

48. The method of claim 1, wherein at least about any of 40%, 50%, 60%, 70%, 80%, and 90% of said cells uptake said agent via passage through said one or more pores.

49. (canceled)

50. (canceled)

51. (canceled)

52. (canceled)

53. (canceled)

54. The method of claim 1, wherein said cells uptake said agent with a cell viability of greater than about any of 50%, 60%, 70%, 80%, and 90%.

55. (canceled)

56. (canceled)

57. (canceled)

58. (canceled)

59. The method of claim 1, wherein said membrane has a maximum thickness substantially equal to a maximum length of said pores.

60. The method of claim 1, wherein said pores have a substantially uniform length and said membrane has a thickness substantially equal to said length.

61. The method of claim 1, wherein said agent comprises any of a deoxyribonucleic acid (DNA), an ribonucleic acid (RNA), a plasmid, a ribonuclear protein complex (RNP), a protein, a peptide, a lipid, a polysaccharide, an oligosaccharide, an antisense oligonucleotide, an aptamer, a nanoparticle, a dye, a non-membrane permeable compound, a membrane impermeable small molecule, and combinations thereof.

62. (canceled)

63. The method of claim 12, further comprising introducing said liquid carrier containing said cells and the agent into said pores via an input chamber having a volume less than about 20% of a volume of the liquid carrier introduced into said pores via said input chamber.

64. (canceled)

65. (canceled)

66. A method of transfecting cells, comprising:
applying a pressure to a liquid carrier containing a plurality of cells at a cell concentration in a range of about 10,000 to about 200,000 cells per microliter so as to cause the liquid carrier and the cells contained therein to pass through a plurality of pores of one or more porous membranes while exposing the cells to an agent so as to transfect at least some of said cells with said agent at a rate greater than about 4 billion cells per minute with a cell viability of at least about 60%;
wherein each of said pores has a maximum cross-sectional dimension in a range of about 7 microns to about 9 microns.

67. (canceled)

68. (canceled)

69. (canceled)

70. (canceled)

71. The method claim 66, wherein said cell viability is at least about 60%.

72. The method of claim 66, wherein said pores have a length in a range of about 7 microns to about 10 microns.

73. A system for parallel cell processing, comprising:
an inlet support element having a plurality of openings for processing a plurality of samples containing a fluid carrier, a plurality of cells and at least one agent to be internalized by the cells;
an outlet support element having a plurality of openings; and
a plurality of porous membranes disposed between said inlet support element and said outlet support element and each having a plurality of pores with a maximum cross-sectional dimension less than about 15 microns;
wherein each of the porous membranes is positioned relative to one of the openings in the inlet support element to receive one of the samples and is positioned relative to one of the openings in the outlet support element to allow at least a portion of the sample passing therethrough to reach said outlet opening.

74. (canceled)

75. (canceled)

76. (canceled)

77. (canceled)

78. (canceled)

79. The system of claim 73, further comprising a plurality of meshes each disposed adjacent one of said porous membranes to provide mechanical support thereto, and wherein said meshes are formed of stainless steel.

80. (canceled)

81. The system of claim 73, wherein said porous membranes comprise a polymeric material.

82. The system of claim 81, wherein said pores are formed in said polymeric material via any of ion tracking etching, laser drilling, plasma etching, or photolithography.

83. The system of claim 73, further comprising at least one pressure applicator coupled to at least one of the openings of the inlet support element for applying a pressure to said samples.

84. The system of claim 73, further comprising at least one inlet chamber disposed upstream of said plurality of porous membranes and having an inlet port for receiving said fluid carrier and an outlet port through which said fluid carrier is introduced onto said porous membranes.

85. The system of claim 84, wherein said input chamber has a volume less than about 20% of a volume of the fluid carrier introduced onto said porous membranes.

86. (canceled)

87. (canceled)

88. The system of claim 84, wherein said at least one inlet chamber comprises a plurality of inlet chambers each of which is disposed upstream of one of said plurality of porous membranes.

89. A process of producing cells for therapy, comprising:
passing a plurality of cells through one or more pores of a membrane comprising a plurality of pores while exposing the cells to a gene-editing complex to cause change in the cells, thereby allowing said gene editing complex to enter at least one of the cells,
wherein each of said pores extends from an input opening to an output opening and has a maximum cross-sectional dimension in a range of about 7 microns to about 9 microns, and
wherein said gene-editing complex is configured to modify said at least one cell for use in gene therapy.

90. The process of claim 89, wherein said gene editing complex comprises a CRISPR gene editing complex.

91. The process of claim 89, wherein said cells comprise hematopoietic stem and progenitor cells (HSPC).

92. The method of claim 91, wherein said gene editing complex is configured to effect a genetic correction in said cells.


SEQUENCE LISTING (16 sequences)

SEQ ID NO: 1 - protein, 7 aa, Simian virus 40
PKKKRKV

SEQ ID NO: 2 - DNA, 42 bp, Unknown
  CRISPR
  repeat_unit (1)..(20): “n”′s refer to the residues of the targeting       domain.
  misc_feature (1)..(20): n is a, c, g, t or u
nnnnnnnnnnnnnnnnnnnnguuuuagagcuaugcuguuuug

SEQ ID NO: 3 - DNA, 79 bp, Unknown
  CRISPR
aacuuaccaaggaacagcauagcaaguuaaaauaaggcuaguccguuaucaacuugaaaaaguggcaccgagucggugc

SEQ ID NO: 4 - DNA, 67 bp, Unknown
  CRISPR
aacagcauagcaaguuaaaauaaggcuaguccguuaucaacuugaaaaaguggcaccgagucggugc

SEQ ID NO: 5 - DNA, 95 bp, Unknown
  CRISPR
  repeat_unit (1)..(19): “n”′s refer to the residues of the targeting       domain.
  misc_feature (1)..(19): n is a, c, g, t or u
nnnnnnnnnnnnnnnnnnnguuuuagagcuagaaauagcaaguuaaaauaaggcuaguccguuaucaacuugaaaaaguggcaccgagucggugc

SEQ ID NO: 6 - DNA, 20 bp, Unknown
  CRISPR
ggccgagaugucucgcuccg

SEQ ID NO: 7 - DNA, 22 bp, Unknown
  CRISPR
ctctcaaaccacagggatcaca

SEQ ID NO: 8 - DNA, 21 bp, Unknown
  CRISPR
ctccatcaccaagagagcctt

SEQ ID NO: 9 - DNA, 20 bp, Unknown
  CRISPR
ggccgagaugucucgcuccg

SEQ ID NO: 10 - DNA, 20 bp, Unknown
  CRISPR
cuugucaaggcuauugguca

SEQ ID NO: 11 - DNA, 27 bp, Unknown
  CRISPR
cgctgaaactgtggctttatagaaatt

SEQ ID NO: 12 - DNA, 24 bp, Unknown
  CRISPR
ggcgtctggactaggagcttattg

SEQ ID NO: 13 - DNA, 26 bp, Unknown
  CRISPR
gcactgaaactgttgctttataggat

SEQ ID NO: 14 - DNA, 26 bp, Unknown
  CRISPR
acggataagtagatattgaggtaagc

SEQ ID NO: 15 - DNA, 21 bp, Unknown
  CRISPR
gtctctttcagttagcagtgg

SEQ ID NO: 16 - DNA, 26 bp, Unknown
  CRISPR
actgcgctgaaactgtggctttatag